(19) United States
(12) Patent Application Publication
(10) Pub. No.: US 2023/0312653 A1
(43) Pub. Date: Oct. 5, 2023

(54) CYCLIC CELL PENETRATING PEPTIDES

(71) Applicant: Entrada Therapeutics, Inc., Boston, MA (US)

(72) Inventors: Patrick Dougherty, Independence, OH (US); Ziqing Qian, Wellesley, MA (US); Mahboubeh Kheirabadi, Natick, MA (US)

(21) Appl. No.: 18/128,669

(22) Filed: Mar. 30, 2023

Related U.S. Application Data
(60) Provisional application No. 63/362,295, filed on Mar. 31, 2022.
(60) Provisional application No. 63/354,382, filed on Jun. 22, 2022.

(51) Int. Cl. C07K 7/64; A61K 47/62; A61K 47/60

(52) U.S. Cl. CPC C07K 7/64; A61K 47/62; A61K 47/60

(57) ABSTRACT

The present disclosure is directed to cell penetrating peptides, including cyclic cell penetrating peptides with high cytosolic delivery efficiency and reduced toxicity that are able to effectively deliver cargo inside a cell to treat a variety of conditions and diseases.

### CROSS-REFERENCE TO RELATED APPLICATIONS

This application claims priority to U.S. provisional patent application No. 63/362,295 which was filed on Mar. 31, 2022, and U.S. provisional patent application No. 63/354,382 which was filed on Jun. 22, 2022, the disclosures of each of which are hereby incorporated by reference in their entireties.

### BACKGROUND

Nucleic acids and their synthetic analogs hold enormous potential as therapeutic agents, especially against targets that are challenging for conventional drug modalities (e.g., missing/defective proteins caused by genetic mutations).

However, a major problem in translating the potential of such therapies to the clinic is their limited ability to gain access to the intracellular compartment when administered systemically. Carrier systems, such as polymers, cationic liposomes or chemical modifications, for example, by the covalent attachment of cholesterol molecules, have been used to facilitate intracellular delivery. Still, intracellular delivery efficiency by these approaches is often low and improved delivery systems to increase efficacy of intracellular delivery have remained elusive.

In the late 1980s, it was discovered that the highly positively charged HIV Tat peptide could translocate across the mammalian cell membrane. Subsequently, other “cell penetrating peptides” (CPPs) have been discovered that are capable of penetrating the cell membrane at low micromolar concentrations without causing significant membrane damage. Qian et al. (2016) “Discovery and Mechanism of Highly Efficient Cyclic Cell-Penetrating Peptides.” Biochem. 55:2601-2612. However, effective cytosolic delivery by many of these CPPs is limited by poor endosomal escape efficiency.

Accordingly, new cell penetrating peptides and compositions comprising peptides with a suitable toxicity profile are needed.

The compositions and methods disclosed herein address these and other needs.

### SUMMARY

One potential strategy to subvert the membrane barrier and deliver drugs into cells is to attach them to “cell-penetrating peptides” (CPP). However, certain residues, such as arginine, which are believed to facilitate cytoplasmic delivery have been implicated as possible contributors to systemic organ toxicity.

Accordingly, new cell penetrating peptides and compositions comprising cell penetrating peptides, for example, with a modified toxicity profile are needed.

Provided herein is a cell penetrating peptide of the general Formula (A):

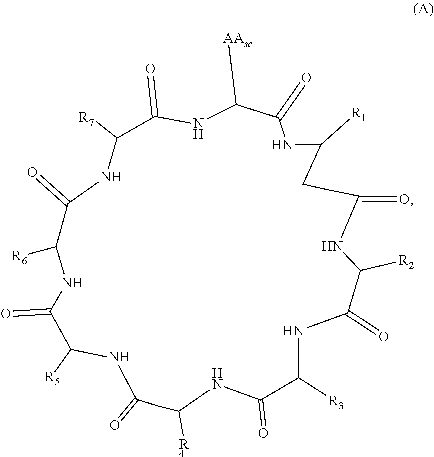

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain;
nₓ is 0 or 1; and
q is 1, 2, 3 or 4.

Provided herein is a cell penetrating peptide of the general Formula (A2):

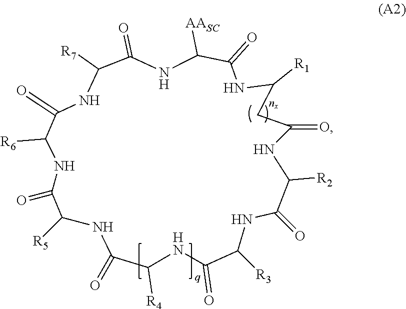

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 1; and
q is 1, 2, 3 or 4.

Provided herein is a cell penetrating peptide of the general Formula (A3):

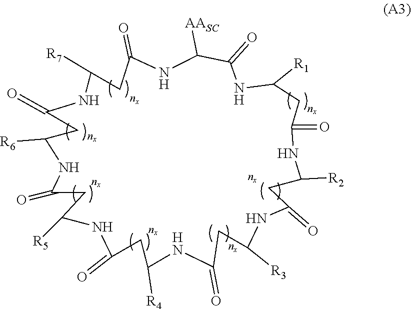

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 0 or 1;
only one nₓ is 1; and
q is 1, 2, 3 or 4.

The compositions and methods disclosed herein address these and other needs.

### DETAILED DESCRIPTION

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid that i mimics the structure and reactivity of a natural amino acid. The non-natural amino acid can be a modified amino acid, and/or amino acid analog that is not one of the 20 common naturally occurring amino acids or a rare natural amino acid such as selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The EP can comprise 1 amino acid residue comprising a side chain comprising a guanidine group, or a protonated form thereof. The EP can comprise 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2 lysine residues. The EP can comprise 3 lysine residues. The EP can comprise 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The EP can comprise 2 amino acid residues with a hydrophobic side chain. The EP can comprise 3 amino acid residues with a hydrophobic side chain. The EP can comprise 4 amino acid residues with a hydrophobic side chain. The amino acid residue with a hydrophobic side chain can be selected from valine, proline, alanine, leucine, isoleucine, and methionine. The amino acid residue with a hydrophobic side chain can be valine, proline or a combination thereof.

The EP can comprise at least one positively charged amino acid residue. The EP can comprises at least one lysine residue. The EP can comprise at least one arginine residue. The EP can comprise at least one lysine and at least one arginine residue. The EP can comprise at least two, at least three or at least four or more lysine residues and/or arginine residues. The EP can comprise two lysine residues and/or arginine residues. The EP can comprise three lysine residues and/or arginine residues. The EP can comprise four lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Cell Penetrating Peptides (CPP)

The cell penetrating peptide (CPP) can comprise 6 to 20 amino acid residues, 6 to 10 amino acid residues or 6 to 8 amino acid residues. The CPP can comprise 6 amino acid residues. The CPP can comprise 7 amino acid residues. The CPP can comprise 8 amino acid residues. The cell penetrating peptide can be a cyclic cell penetrating peptide (cCPP). In embodiments, the cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

Each amino acid in the cCPP may be a natural or non-natural amino acid. The term “amino acid” refers to a compound having an amino group and a carboxylic acid group. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

As used herein, the term “amino acid” refers to compounds having an amino group and a carboxylic acid group. Most amino acids (except for glycine) also have a side chain. As used herein, “amino acid side chain” or “side chain” refers to the characterizing substituent bound to the α-carbon of the amino acid.

An “α-amino acid” is an amino acid in which the amino group is attached to the first (alpha) carbon adjacent to the carboxylic acid group, such that the carbon atom of the carbonyl is separated from the nitrogen atom of the amino group by one carbon atom. A “β-amino acid” is an analog of an α-amino acid in which the amino group is attached to the second (beta) carbon, rather than the alpha-carbon, such that the carbon atom of the carbonyl is separated from the nitrogen atom of the amino group by two carbon atoms. Examples of β-amino acids include, but are not limited to β-alanine and β-homophenylalanine. An “uncharged” amino acid is an amino acid that does not have a charge at a physiological pH (between 5.0 and 7.0). It is noted that histidine can exist in neutral or positively charged forms at physiological pH.

A side chain that does not comprise an aryl or heteroaryl group, can be referred to herein as a “non-aryl” side chain. In embodiments, the side chain that does not comprise an aryl or heteroaryl group can be uncharged and is referred to herein as an uncharged, non-aryl side chain. Amino acids with uncharged non-aryl amino side chains include, but are not limited to, histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allo-isoleucine | Aile | aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| methionine | Met (M) | met (m) |
| 3-(2-naphthyl)-alanine | Nal (Φ) | nal (ϕ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodi- | F₂Pmp (Λ) | f₂pmp |
| fluoromethyl)phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothieny1)-alanine | Bta | bta |

*single letter abbreviations: when shown in capital letters herein it indicates the L-amino acid form, when shown in lower case herein it indicates the D-amino acid form.

One or two amino acids in the CPP can have no side chain. In embodiments, all amino acids in the CPP have a side chain. As used herein, when no side chain is present, the amino acid has two hydrogen atoms on the carbon atom(s) (e.g., —CH₂—) linking the amine and carboxylic acid of the amino acid residue. The amino acid having no side chain can be glycine or β-alanine.

The cCPP can comprise from 6 to 20, from 6 to 10, or from 6 to 8 amino acid residues, wherein: (i) at least one amino acid can be glycine, β-alanine, serine, histidine or 4-aminobutyric acid; (ii) at least one amino acid can have a side chain comprising an aryl or heteroaryl group; and (iii) at least one amino acid has a side chain comprising a guanidine group, or a protonated form thereof.

In embodiments, one amino acid of the CPP can be glycine, β-alanine, serine, histidine, or 4-aminobutyric acid. In embodiments, two amino acid can be, independently, glycine, β-alanine, serine, histidine or 4-aminobutyric acid. In embodiments, three amino acid can be glycine, β-alanine, serine, histidine, or 4-aminobutyric acid.

In embodiments, one amino acid of the CPP can have a side chain comprising an aryl or heteroaryl group. In embodiments, two amino acids of the CPP can have a side chain comprising an aryl or heteroaryl group. In embodiments, three amino acid of the CPP can have a side chain comprising an aryl or heteroaryl group.

In embodiments, one amino acid of the CPP can have a side chain that does not comprise an aryl or heteroaryl group, referred to herein as a “non-aryl” side chain. In embodiments, the side chain that does not comprise an aryl or heteroaryl group can be uncharged and is referred to herein as an uncharged, non-aryl side chain. In embodiments, two amino acids of the CPP can have an uncharged, non-aryl side chain. In embodiments, three amino acid of the CPP can have an uncharged, non-aryl side chain. Amino acids with uncharged non-aryl amino side chains include, but are not limited to, histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

In embodiments, one amino acid of the CPP has a side chain comprising a guanidine group, or a protonated form thereof. In embodiments, two amino acid of the CPP can have a side chain comprising a guanidine group, or a protonated form thereof. In embodiments, three amino acid of the CPP can have a side chain comprising a guanidine group, or a protonated form thereof. In embodiments, four amino acid of the CPP can have a side chain comprising a guanidine group, or a protonated form thereof.

The cCPP can comprise 1 or 2 amino acid residues selected from uncharged non-aryl amino acids residues.

The cCPP can comprise 2 contiguous amino acids with hydrophobic side chains The cCPP can comprise 3 contiguous amino acids with hydrophobic side chains.

### Glycine and Related Amino Acid Residues

The cCPP can comprise 1, 2, 3, 4, 5, or 6 glycine residues. The cCPP can comprise 1 glycine residue. The cCPP can comprise 2 glycine residues. The cCPP can comprise 3 glycine residues. The cCPP can comprise 4 glycine residues.

In embodiments, none of the glycine residues in the cCPP are contiguous. In embodiments, each glycine residue in the cCPP spaced apart from another glycine residue by an amino acid residue that is not glycine. In embodiments, two glycine residues in the cCPP can be contiguous

Amino Acid Side Chains with an Aromatic or Heteroaromatic Group

The cCPP can comprise 2 or 3 amino acid residues independently having a side chain comprising an aromatic or heteroaromatic group. The cCPP can comprise 2 amino acid residues independently having a side chain comprising an aromatic or heteroaromatic group. The cCPP can comprise 3 amino acid residues independently having a side chain comprising an aromatic or heteroaromatic group.

The cCPP can comprise 2 or 3, 4, 5 or 6 amino acid residues independently having a side chain comprising an aromatic group. The cCPP can comprise 2 amino acid residues independently having a side chain comprising an aromatic group. The cCPP can comprise 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, p-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, homonaphthylalanine tryptophan, or tyrosine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), bis(homonaphthylalanine), tryptophan or tyrosine, each of which is optionally substituted with one or more substituents. The amino acid residue having a side chain comprising an aromatic group can each be independently a residue of phenylalanine, naphthylalanine, or tyrosine each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

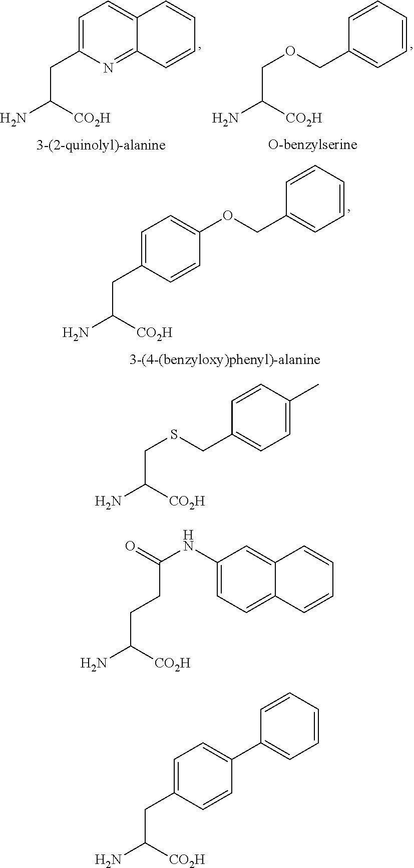 [SMILES: NC(Cc1ccc2ccccc2n1)C(=O)O ; NC(COCc1ccccc1)C(=O)O ; NC(Cc1ccc(OCc2ccccc2)cc1)C(=O)O ; Cc1ccc(CSCC(N)C(=O)O)cc1 ; NC(CCC(=O)Nc1ccc2ccccc2c1)C(=O)O ; NC(Cc1ccc(-c2ccccc2)cc1)C(=O)O]

S-(4-methylbenzyl)cysteine, N⁵-(naphthalen-2-yl)glutamine, 3-(1,1′-biphenyl-4-yl)-alanine, and

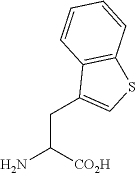 [SMILES: NC(Cc1csc2ccccc12)C(=O)O]

3-(3-benzothienyl)-alanine, wherein the H on the N-terminus and/or the H on the C-terminus are replaced by a peptide bond.

Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The amino acid side chain can be substituted with one or more substituents selected from halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

### Amino Acid Residues Having a Side Chain Comprising a Guanidine Group, or Protonated Form Thereof

As used herein, guanidine refers to the structure:

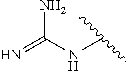

As used herein, a protonated form of guanidine refers to the structure:

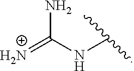

The disclosure relates to a cCPP comprising from 6 to 20 amino acids residues, 6 to 10 amino acid residues or 6 to 8 amino acid residues, wherein: (i) at least one amino acid has a side chain comprising a guanidine group, or a protonated form thereof, (ii) at least one amino acid residue is an uncharged non-aryl side chain; and (iii) at least two amino acids residues independently have a side chain comprising an aromatic or heteroaromatic group. In embodiments, the amino acids with an uncharged non-aryl amino side chain is selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine. In embodiments, at least one amino acid is a β-amino acid. In embodiments, at least one amino acid is serine or histidine. In embodiments, at least one amino acid is a β-amino acid and at least two amino acids are independently serine or histidine.

At least two amino acids residues can have no side chain. As used herein, when no side chain is present, the amino acid residue have two hydrogen atoms on the carbon atom(s) (e.g., —CH₂—) linking the amine and carboxylic acid.

The cCPP can comprise 1, 2, 3, or 4, amino acid residues independently having a side chain comprising a guanidine group, or a protonated form thereof. The cCPP can comprise 1 amino acid residues having a side chain comprising a guanidine group, or a protonated form thereof. The cCPP can comprise 2 amino acid residues independently having a side chain comprising a guanidine group, or a protonated form thereof. The cCPP can comprise 3 amino acid residues independently having a side chain comprising a guanidine group, or a protonated form thereof. The cCPP can comprise 4 amino acid residues independently having a side chain comprising a guanidine group, or a protonated form thereof.

The amino acid residues can independently have the side chain comprising the guanidine group, or the protonated form thereof that are not contiguous. Two amino acid residues can independently have a side chain comprising a guanidine group, or the protonated form thereof are not contiguous. The non-contiguous amino acid residues can have the same stereochemistry. The non-contiguous amino acids can have alternating stereochemistry.

The amino acid residues independently having the side chain comprising the guanidine group, or the protonated form thereof, can be L-amino acids. The amino acid residues independently having the side chain comprising the guanidine group, or the protonated form thereof, can be D-amino acids. The amino acid residues independently having the side chain comprising the guanidine group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a residue of asparagine, aspartic acid, glutamine, glutaminc acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

The cCPP can have no D amino acids. The cCPP can comprise at least one D amino acid. The cCPP can comprise 1, 2, or 3 D amino acids. The cCPP can comprise 2 or 3 contiguous amino acids having alternating D and L chirality. The cCPP can comprise three contiguous amino acids having the same chirality. The cCPP can comprise two contiguous amino acids having the same chirality. Two of the amino acids can have the opposite chirality. Two amino acids having the opposite chirality can be adjacent to each other. Three amino acids can have alternating stereochemistry relative to each other. Three amino acids having the alternating chirality relative to each other can be adjacent to each other. Two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids.

The cCPPs can comprise the following sequences: L/D-X-L/D; L/D-X-L/D-X; L/D-X-L/D-X-L/D; L-X-L; L-X-L-X; L-X-L-X-L; D-X-D; D-X-D-X; or D-X-D-X-D, wherein L/D indicates that the amino acid can be and L-amino acid or a D-amino acid and X is an achiral amino acid. The achiral amino acid can be glycine.

The cCPP can comprise the structure of Formula (A):

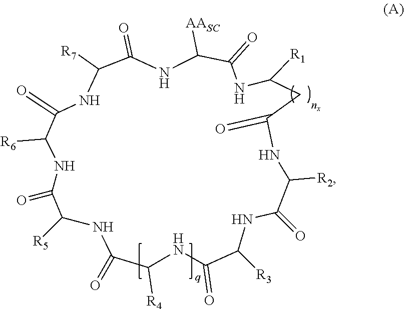

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine; at least two of R₄, R₅, R₆, or R₇ are independently a side chain of serine or histidine;
AA_{SC} is an amino acid side chain;
nₓ is 0 or 1; and
q is 1, 2, 3 or 4.

The cCPP can comprise the structure of Formula (A2):

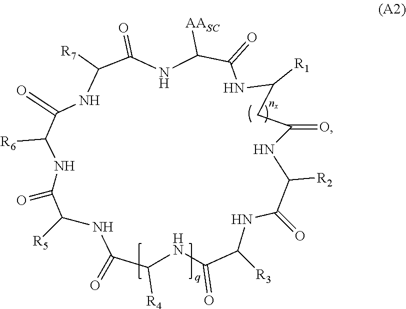

or a protonated form thereof,

wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 1; and
q is 1, 2, 3 or 4.

The cCPP can comprise the structure of (A3):

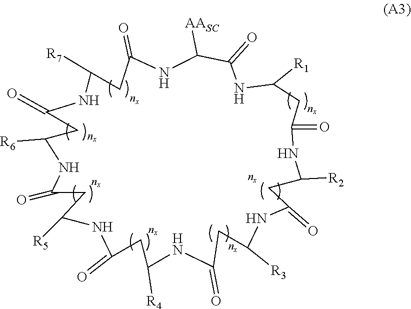

R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 0 or 1;
only one nₓ is 1; and
q is 1, 2, 3 or 4.

In embodiments, the CPP is of the general Formula A, wherein at least one of R₁, R₂, or R₃ is H. In embodiments, the CPP is of the general Formula A, wherein at least one of R₁, R₂, or R₃ is a side chain of phenylalanine. In embodiments, the CPP is of the general Formula A, wherein at least two of R₁, R₂, or R₃ are a side chain of naphthylalanine.

In embodiments, the CPP is of the general Formula A, wherein at least one of R₁, R₂, or R₃ is independently a side chain of phenylalanine or naphthylalanine.

In embodiments, the CPP is of the general Formula A, wherein q is 1. In embodiments, the CPP is of the general Formula A, wherein q is 1 and nₓ is 1. In embodiments, the CPP is of the general Formula A, wherein q is 1 and nₓ is 0.

In embodiments, the CPP is of the general Formula B, wherein q is 1.

In embodiments, the CPP is of the general Formula C, wherein

In embodiments, at least two of R₄, R₅, R₆, or R₇ are independently a side chain of serine or histidine.

In embodiments, at least two of R₄, R₅, R₆, or R₇ are independently a side chain of arginine.

In embodiments, at least one of R₄, R₅, R₆, R₇ are independently a uncharged, non-aryl side chain of an amino acid. In embodiments, at least two of R₄, R₅, R₆, or R, are independently side chains of an uncharged non-aryl amino acid. at least two of R₄, R₅, R₆, or R₇ are independently side chains of an uncharged non-aryl amino acid selected from histidine, threonine, serine, leucine, isoleucine, valine, neopentylglycine, alanine, homoalanine, homoserine, 3-(4-Thiazolyl)-alanine, 3-(4-furanyl)-alanine, and 3-(4-thienyl)-alanine.

In embodiments, at least one of R₄, R₅, R₆, R₇ are independently H.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aryl amino acids. In embodiments, at least two charged amino acids of the cyclic peptide are arginine. In embodiments, at least two aromatic, hydrophobic amino acids of the cyclic peptide are phenylalanine, naphthylalanine (3-naphth-2-yl-alanine) or a combination thereof. In embodiments, at least two uncharged, non-aryl amino acids of the cyclic peptide are glycine. In embodiments, two of the uncharged amino acids are serine, histidine or a combination thereof.

In embodiments, the CPP is of the general Formula (A), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine;
AA_{SC} is an amino acid side chain;
nₓ is 0 or 1; and
q is 1, 2, 3 or 4.

In embodiments, the CPP is of the general Formula (A), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine; at least two of R₄, R₅, R₆, or R₇ are independently a side chain of serine or histidine;
AA_{SC} is an amino acid side chain;
nₓ is 0 or 1; and
q is 1.

In embodiments, the CPP is of the general Formula (A), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain;
nₓ is 1; and
q is 1.

In embodiments, the CPP is of the general Formula (A), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine;
AA_{SC} is an amino acid side chain;
nₓ is 0 or 1; and
q is 1.

In embodiments, the CPP is of the general Formula (A), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of histidine;
AA_{SC} is an amino acid side chain;
nₓ is 0 or 1; and
q is 1.

In embodiments, the CPP is of the general Formula (A2), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 1; and
q is 1.

In embodiments, the CPP is of the general Formula (A2), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine; at least two of R₄, R₅, R₆, or R₇ are independently a side chain of serine or histidine;
AA_{SC} is an amino acid side chain; and
nₓ is 1; and
q is 1, 2, or 4. In embodiments, the CPP is of the general Formula (A2), wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain; and
nₓ is 1; and
q is 1.

The cCPP can comprise the structure of Formula (I):

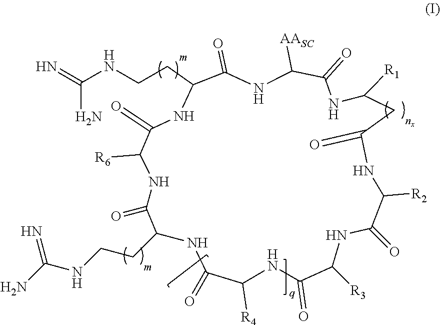

or a protonated form thereof,
wherein:
R₁, R₂, and R₃ can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
nₓ is 1; and
each m is independently an integer 0, 1, 2, or 3.

In embodiments, the CPP is of the general Formula I, wherein
R₁, R₂, and R₃ can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently H or side chain of serine or histidine;
AA_{SC} is an amino acid side chain;
q is 1, 2, 3 or 4;
nₓ is 1; and
each m is independently an integer 0, 1, 2, or 3.

In embodiments, the CPP is of the general Formula I, wherein
R₁, R₂, and R₃ can each independently be H or an amino acid residue having a side chain comprising an aromatic or heteroaromatic group;
at least two of R₁, R₂, and R₃ is an aromatic or heteroaromatic side chain of an amino acid;
R₄ and R₆ are independently a side chain of serine or histidine;
AA_{SC} is an amino acid side chain;
q is 1, 2, 3 or 4;
nₓ is 1; and
each m is independently an integer 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂-naphthyl. R₁, R₂, and R₃ can each independently be H or —CH₂Ph.

R₁, R₂, and R₃ can each independently be the side chain of phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, p-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ and R₂ can be side chains of phenylalanine and R₃ can be a side chain of 2-naphthylalanine.

R₄ can be H. R₄ can be H or the side chain of an amino acid in Table 1. R₄ can be a residue of an uncharged non-aryl amino acid. In embodiments, R₄ is a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine. R₄ can be a side chain of serine. R₄ can be a side chain of histidine.

R₆ can be H or the side chain of an amino acid in Table 1. R₆ can be a residue of an uncharged non-aryl amino acid. In embodiments, R₆ is a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine. R₆ can be a side chain of serine. R₆ can be a side chain of histidine.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R, can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

nₓ can be 0. n_{X} can be 1.

The cCPP of Formula (A) or (A2) can comprise the structure of Formula (I)

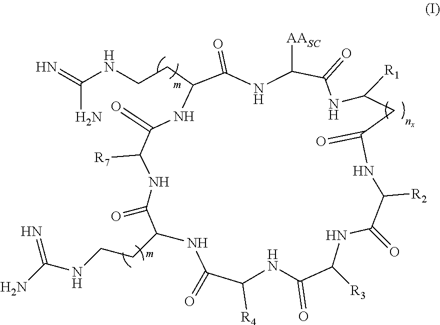

or protonated form thereof, wherein AA_{SC}, R₁, R₂, R₃, R₄, R₇, m and q are as defined herein for Formula (A) or (A2).

The cCPP of Formula (A) or (A2) can comprise the structure of Formula (I′)

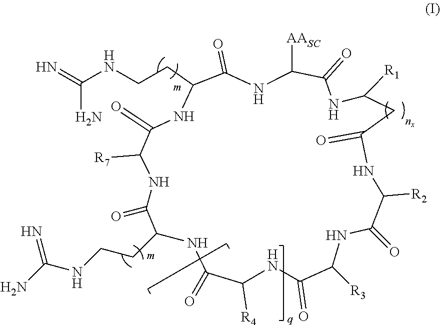

or protonated form thereof, wherein at least one of R₁, R₂, R₃, R₄, or R₇, are a B-amino acid, AA_{SC}, q, and m are as defined herein for Formula (A) or (A2). In embodiments, at least one of R₁, R₂, R₃ is a side chain of (B-hF). In embodiments, at least one of R₄, or R₇ is a side chain of (B-alanine).

The cCPP of Formula (A) or (A2) can comprise the structure of Formula (I-a) or Formula (I-b):

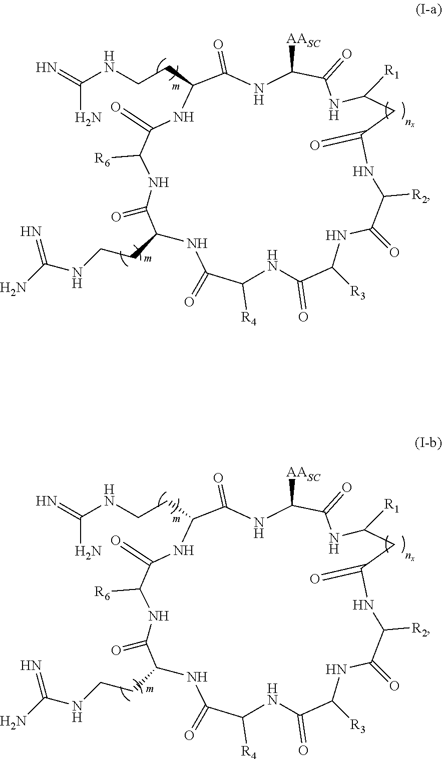

or protonated form thereof, wherein AA_{SC}, R₁, R₂, R₃, R₄, R₇, m and nₓ are as defined herein for Formula (A) or (A2).

The cCPP of Formula (A) can comprise the structure of Formula (I4a), (I-4b), or (I-4c):

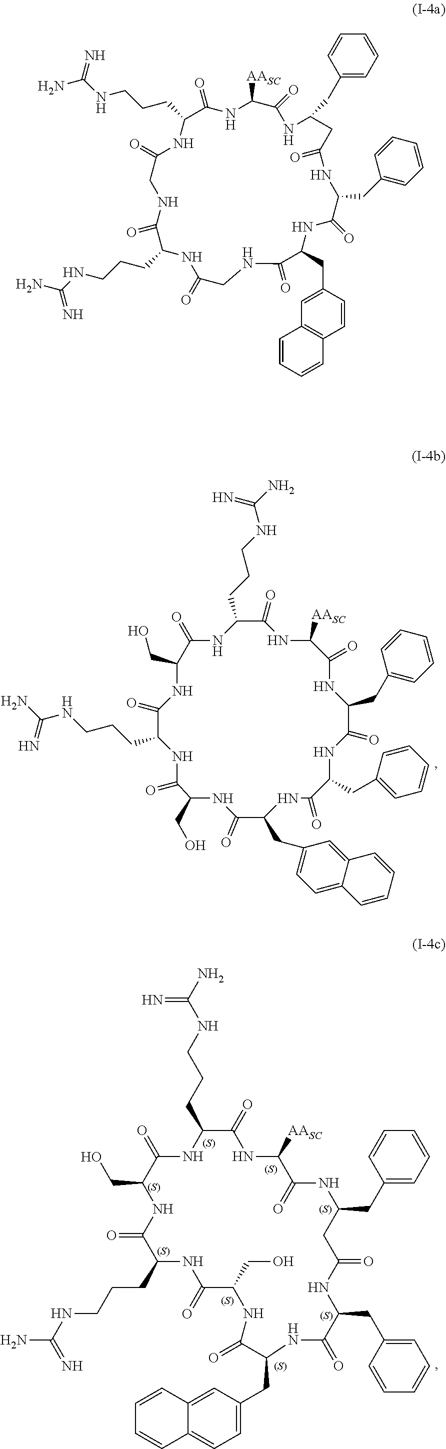

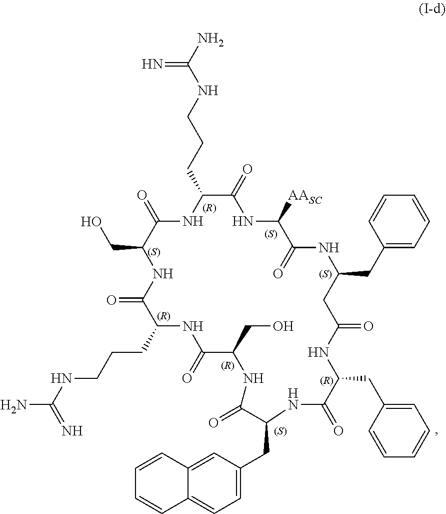

or a protonated form thereof,
wherein AA_{SC} is as defined herein for Formula (A).

The cCPP can comprise one of the following sequences: βhFfΦGrGr; βhFfΦSRSR; or FfΦSrSr. The cCPP can comprise one of the following sequences: βhFfΦGrGrQ; βhFfΦSRSRQ; or FfΦSrSrQ.

The disclosure also relates to a cCPP having the structure of Formula (II):

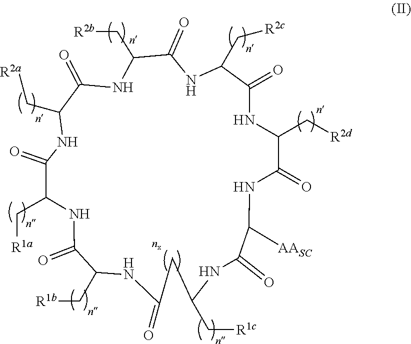

wherein:
AA_{SC} is an amino acid side chain;
R^{1a}, R^{1b}, and R^{1c} are independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is guanidine or a protonated form thereof,
each n″ is independently an integer 0, 1, 2, 3, 4, or 5;
each n′ is independently an integer from 0, 1, 2, or 3;
nₓ is 0 or 1; and
if n′ is 0 then R^{2a}, R^{2b}, R^{2c} or R² is absent.

One or two of R^{2a}, R^{2b}, R^{2c} or R^{2d} are guanidine, or a protonated form thereof, and the remaining of R^{2a}, R^{2b}, R^{2c} or R^{2d} are uncharged non-aryl amino acid side chains. Amino acids with uncharged non-aryl side chains include, but are not limited to, histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

Each of R^{2a}, R^{2b}, R^{2c} and R^{2d} can independently be serine, homo-serine, threonine, allo-threonine, histidine, or 1-methylhistidine.

AA_{SC} can be

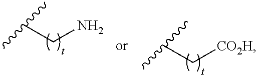

wherein t can be an integer from 0 to 5. AA^{SC} can be

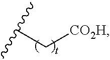

wherein t can be an integer from 0 to 5. t can be 1 to 5. t is 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3.

Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

Each nₓ can independently be 0 or 1. nₓ can be 0. nₓ can be 1.

The cCPP of Formula (A2) can have the structure of Formula (II-1):

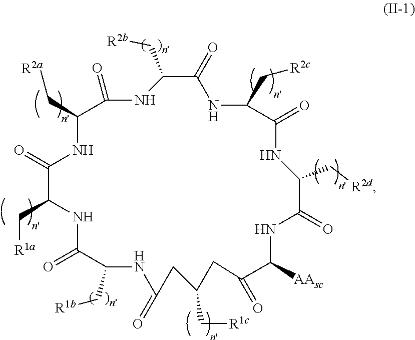

wherein R^{1a}, R^{1b}, R^{1c}, R^{2a}, R^{2b}, R^{2c}, R^{2d}, AA_{SC}, n′, n″, and nₓ are as defined herein for Formula (A2).

The cCPP of Formula (A2) can have the structure of Formula (IIa):

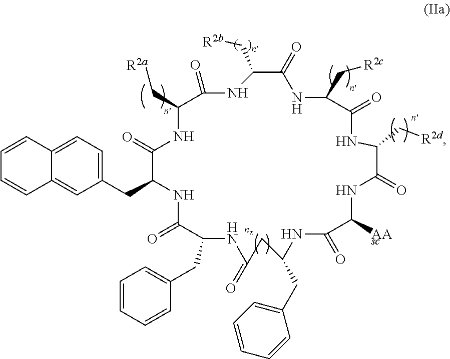

wherein R^{1a}, R^{1b}, R^{1c}, R^{2a}, R^{2b}, R^{2c}, R^{2d}, AA_{SC}, n′, and nₓ are as defined herein herein for Formula (A2).

The cCPP of formula (A2) can have the structure of Formula (III):

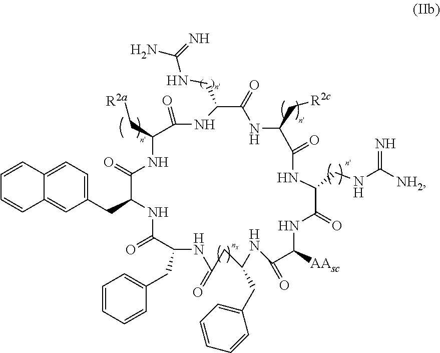

wherein R^{2a}, R^{2b}, AA_{SC}, n′, and nₓ are as defined herein herein for Formula (II).

The cCPP can have the structure of Formula (III):

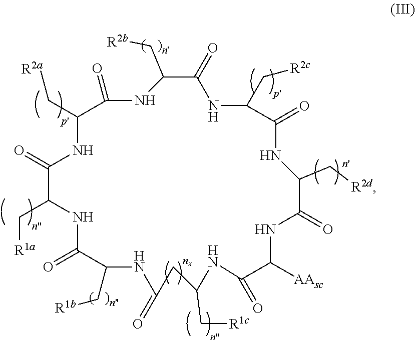

wherein:
AA_{SC} is an amino acid side chain;
R^{1a}, R^{1b}, and R^{1c} are independently a 6- to 14-membered aryl or a 6- to 14-membered heteroaryl;
R^{2a} and R^{2c} are independently H, or uncharged non-aryl amino acid side chain;
R^{2b} and R^{2d} are independently guanidine or a protonated form thereof,
each n″ is independently an integer from 1 to 3;
each n′ is independently an integer from 1 to 5;
each nₓ is 0 or 1; and
each p′ is independently 0 or 1.

The cCPP of Formula (A3) can have the structure of Formula (III-1):

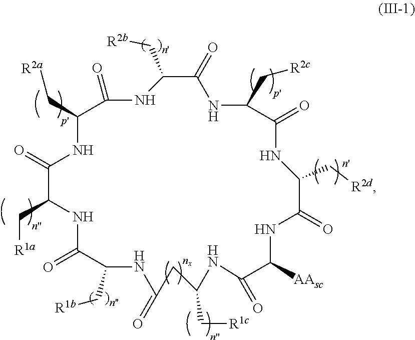

wherein:
AA_{SC}, R^{1a}, R^{1b}, R^{1c}, R^{2a}, R^{2c}, R^{2b}, R^{2d} n′, n″, nₓ, and p′ are as defined herein for Formula (III).

The cCPP of Formula (III) can have the structure of Formula (IIIa):

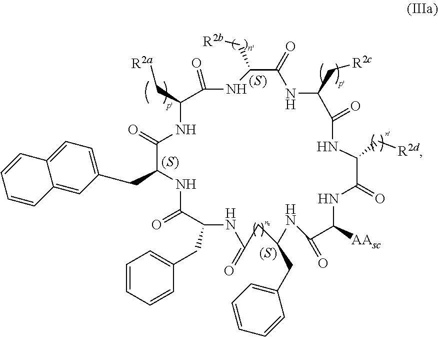

wherein:
AA_{SC}, R^{2a}, R^{2c}, R^{2b}, R^{2d} n′, n″, nₓ, and p′ are as defined herein for Formula (III).

In Formulas (III), (III-1), and (IIIa), R^{2a} and R^{2c} can be H. R^{2a} and R^{2c} can be H and R^{2b} and R^{2d} can each independently be guanidine or protonated form thereof. R^{2a} can be H. R^{2b} can be H. p′ can be 0. R^{2a} and R^{2c} can be H or uncharged non-aryl amino acid side chain and each p′ can be 0, or 1.

In Formulas (III), (III-1), and (IIIa), R^{2a} and R^{2c} can be H or or uncharged non-aryl amino acid side chain, R^{2b} and R^{2d} can each independently be guanidine or protonated form thereof, n″ can be 2 or 3, and each p′ can be 0, or 1.

p′ can 0. p′ can 1.

nₓ can be 0. nₓ can be 1.

The cCPP can have the structure:

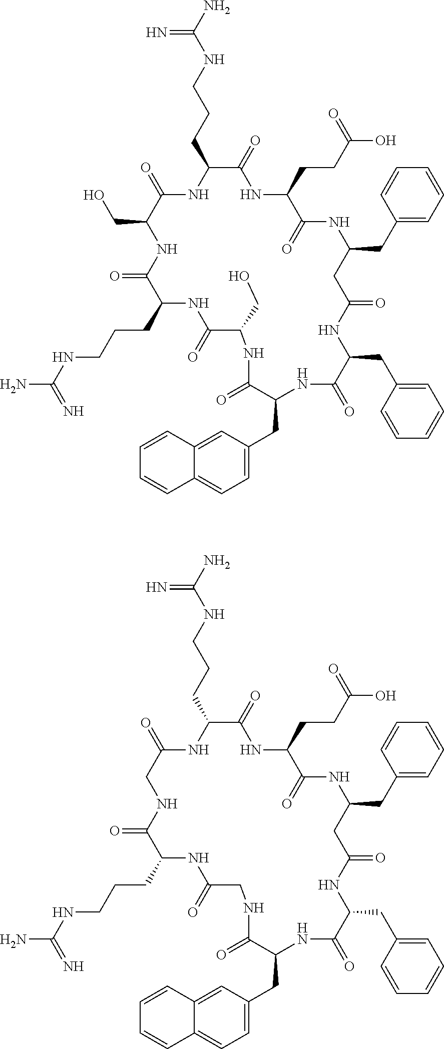 [SMILES: N=C(N)NCCC[C@@H]1NC(=O)[C@H](CO)NC(=O)[C@H](CCCNC(=N)N)NC(=O)[C@H](CO)NC(=O)[C@H](Cc2ccc3ccccc3c2)NC(=O)[C@H](Cc2ccccc2)NC(=O)C[C@H](Cc2ccccc2)NC(=O)[C@H](CCC(=O)O)NC1=O ; N=C(N)NCCC[C@H]1NC(=O)CNC(=O)[C@H](Cc2ccc3ccccc3c2)NC(=O)[C@@H](Cc2ccccc2)NC(=O)C[C@H](Cc2ccccc2)NC(=O)[C@H](CCC(=O)O)NC(=O)[C@@H](CCCNC(=N)N)NC(=O)CNC1=O]

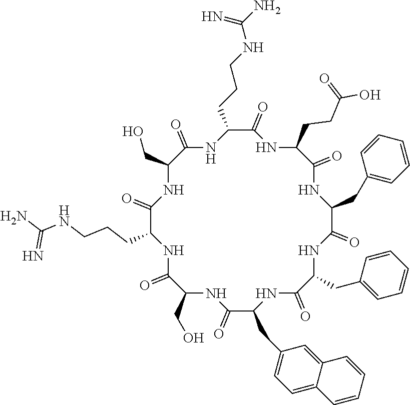 [SMILES: N=C(N)NCCC[C@H]1NC(=O)[C@H](CO)NC(=O)[C@@H](CCCNC(=N)N)NC(=O)[C@H](CO)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (A) can be selected from:

CPP Sequence

(βhFfΦGrGrQ)
(βhFfΦSrSrQ)
(βhFfΦHrHrQ)
(βhFFΦSRSRQ)
(βhFFΦGRGRQ)
(βhFFΦHRHRQ)
(FfΦSrSrQ)
(FfΦHrHrQ)

The cCPP can comprise the structure of Formula (D)

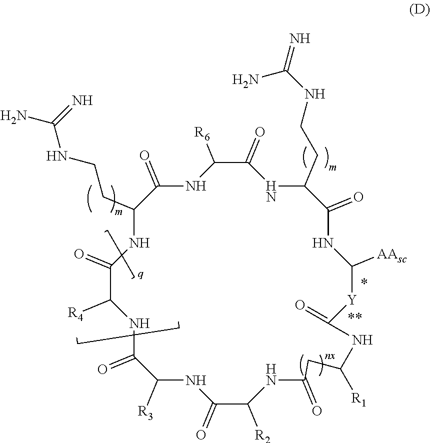

or a protonated form thereof,
wherein:
R¹, R², and R³ can each independently be H or an amino acid residue having a side chain comprising an aromatic group;
at least one of R¹, R², and R³ is an aromatic or heteroaromatic side chain of an amino acid;
R⁴ and R⁶ are independently H or an uncharged non-aryl amino acid side chain;
AA_{SC} is an amino acid side chain;
Y is

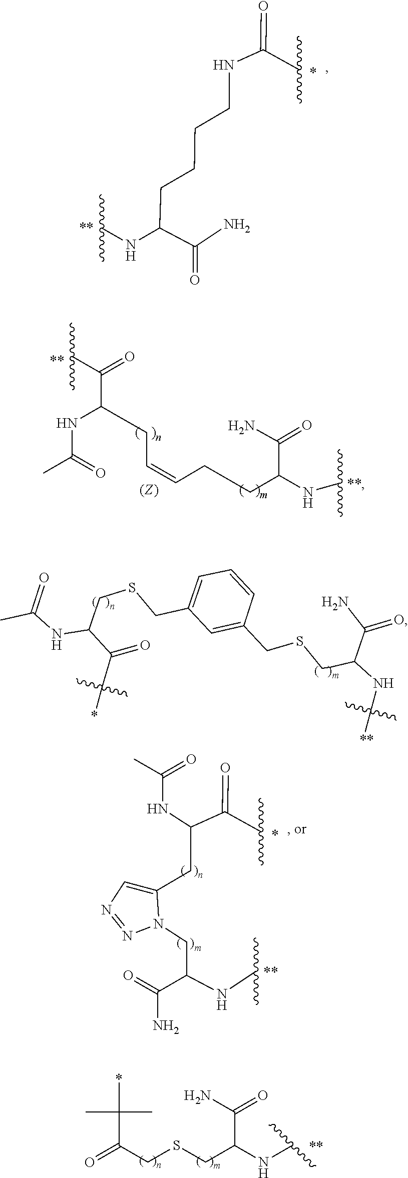

q is 1, 2, 3 or 4;
   each m is independently an integer 0, 1, 2, or 3,
   each n is independently an integer 0, 1, 2, or 3,
   nₓ is 0 or 1; and
   and ** denotes points of attachment.

The cCPP of Formula (D) can have the structure of Formula (D-I):

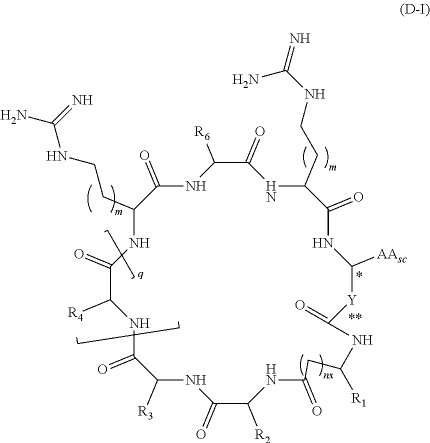

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
q is 1, 2, 3 or 4;
nₓ is 0 or 1;
each m is independently an integer 0, 1, 2, or 3,
Y is

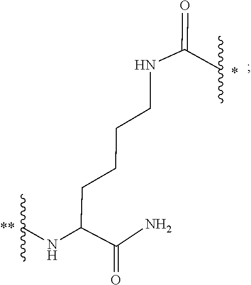 [SMILES: *NC(CCCCNC(*)=O)C(N)=O]

and
and ** denotes points of attachment.

The cCPP of Formula (D) can have the structure of Formula (D-II):

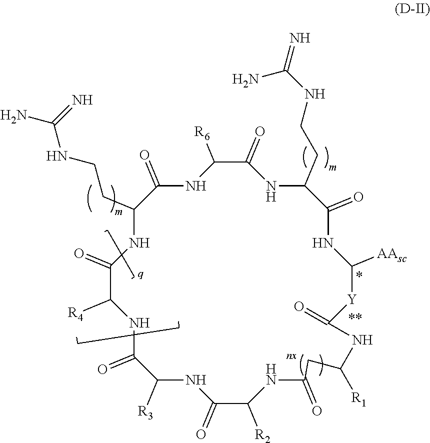

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
q is 1, 2, 3 or 4;
each m is independently an integer 0, 1, 2, or 3,
each n is independently an integer 0, 1, 2, or 3,
nₓ is 0 or 1,
Y is

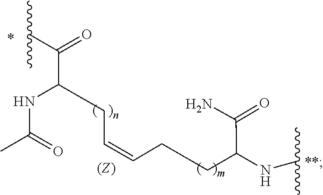

and
and ** denotes points of attachment.

The cCPP of Formula (D) can have the structure of Formula (D-III):

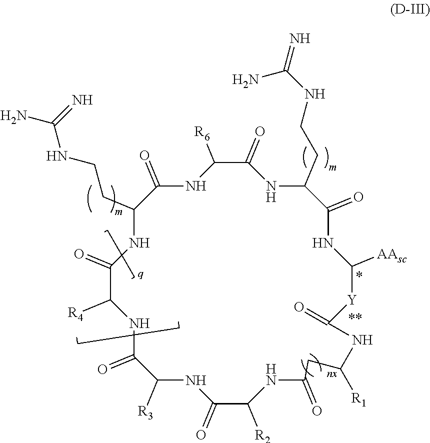

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
q is 1, 2, 3 or 4;
each m is independently an integer 0, 1, 2, or 3;
each n is independently an integer 0, 1, 2, or 3;
each nₓ is 0 or 1; and
Y is

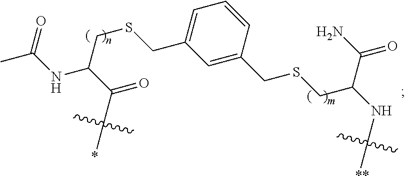

and
and ** denotes points of attachment.

The cCPP of Formula (D) can have the structure of Formula (D-IV):

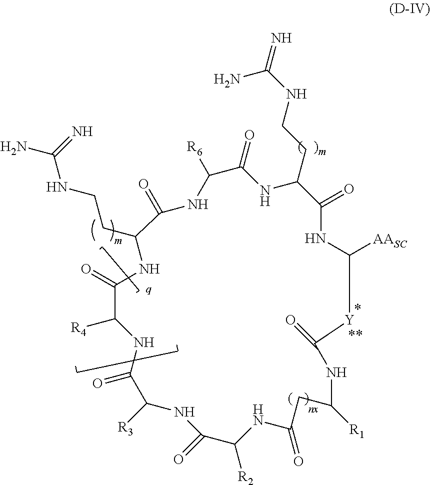

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
q is 1, 2, 3 or 4;
each m is independently an integer 0, 1, 2, or 3,
each nₓ is 0 or 1;

Y is

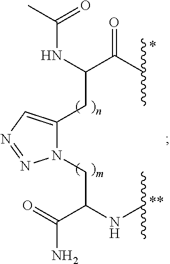

and
and ** denotes points of attachment.

The cCPP of Formula (D) can have the structure of Formula (D-V):

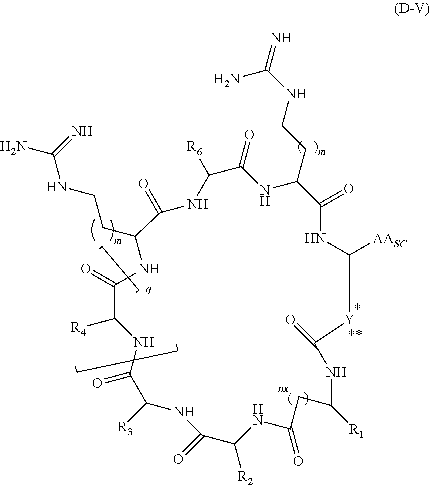

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an uncharged non-aryl amino acid side chain;
AA_{SC} is an amino acid side chain;
q is 1, 2, 3 or 4;
each m is independently an integer 0, 1, 2, or 3;
each nₓ is 0 or 1;

Y is

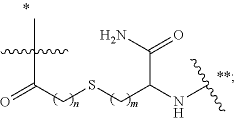

and
and ** denotes points of attachment.
AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g., Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″- subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″—, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₃) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of p-alanine, glycine, lysine, 4-aminobutyric acid, 5-aminopentanoic acid, 6-aminohexanoic acid or combinations thereof, and (ii) —(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

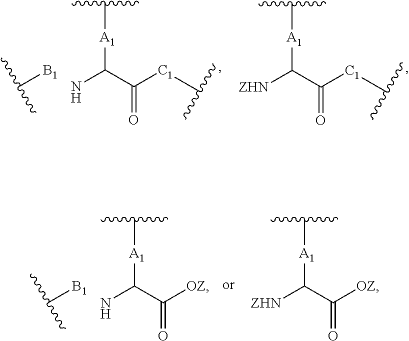

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

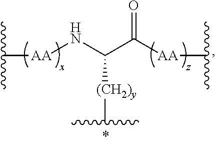

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

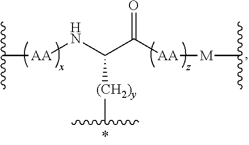

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

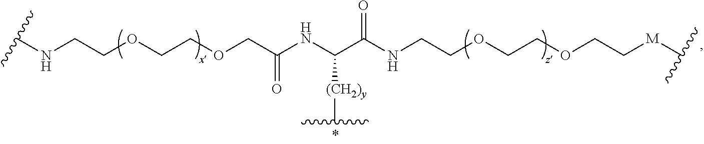

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

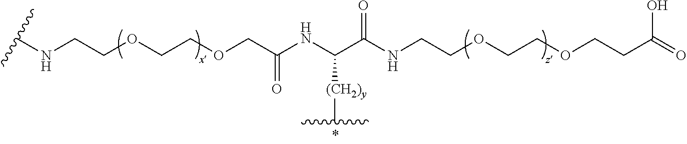

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

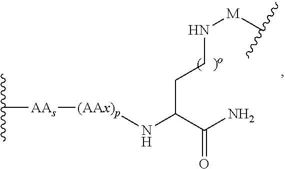

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

The linker can have a structure:

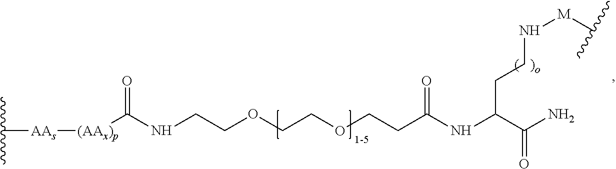

wherein
M is a group that conjugates L to a cargo, for example, an oligonucleotide;
AAₛ is a side chain or terminus of an amino acid on the cCPP;
each AAₓ is independently an amino acid residue;
o is an integer from 0 to 10; and
p is an integer from 0 to 5.

M can comprise an alkylene, alkenylene, alkynylene, carbocyclyl, or heterocyclyl, each of which is optionally substituted. M can be selected from:

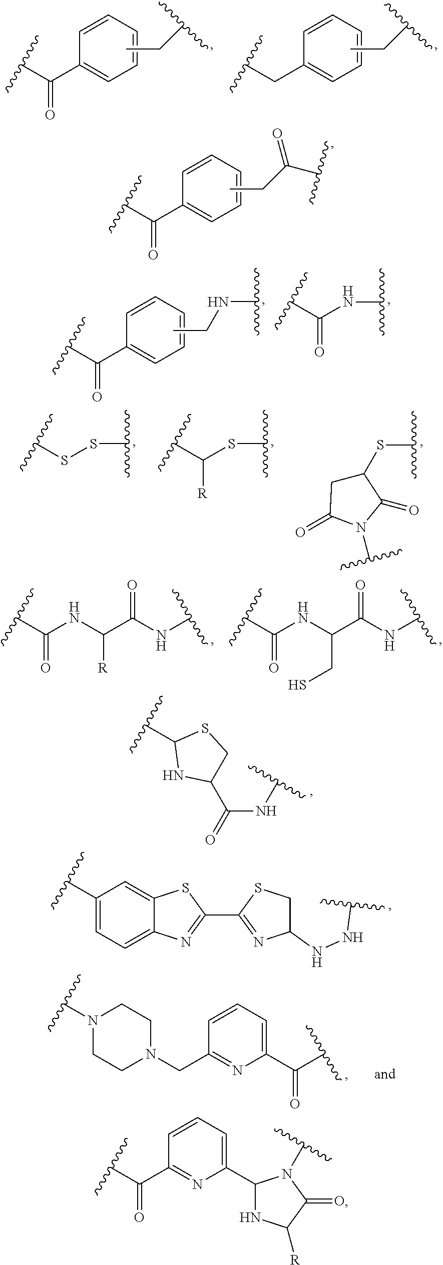

wherein
R is alkyl, alkenyl, alkynyl, carbocyclyl, or heterocyclyl.
M can be selected from:

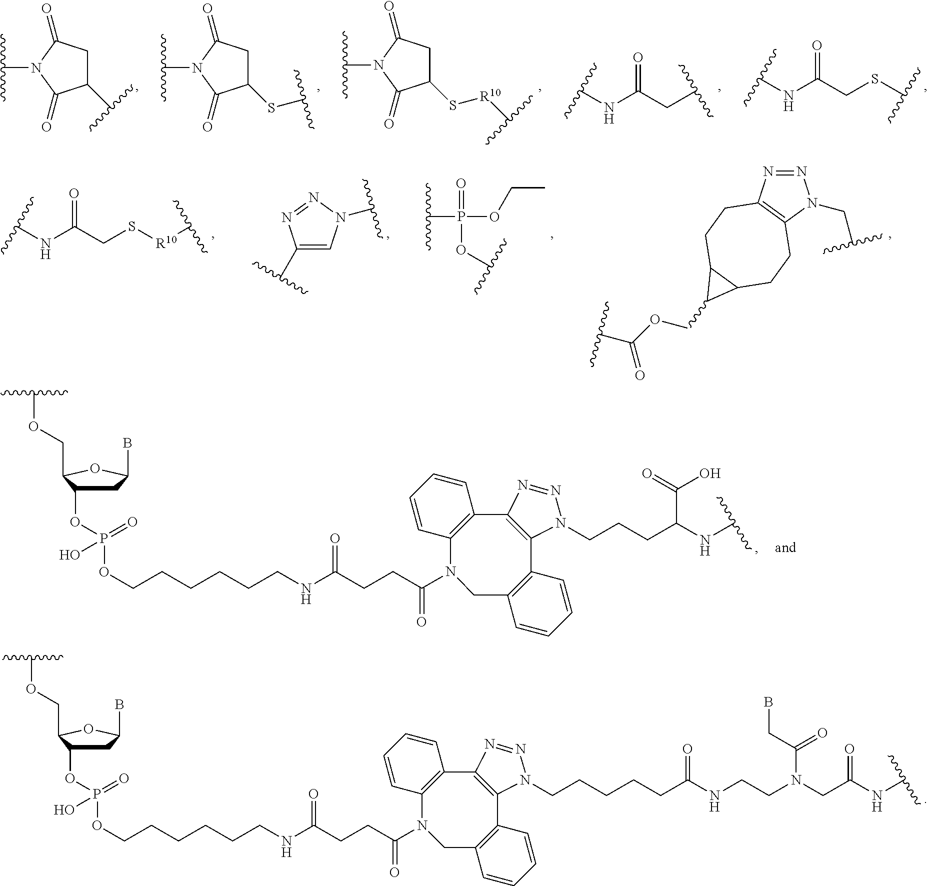

wherein: R¹⁰ is alkylene, cycloalkyl, or

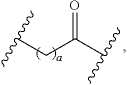

wherein a is 0 to 10.

M can be

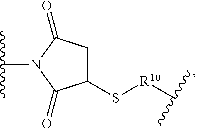

### R¹⁰ can be

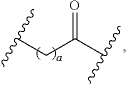

### and a is 0 to 10. M can be.

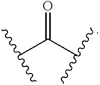 [SMILES: *C(*)=O]

M can be a heterobifunctional crosslinker, e.g.,

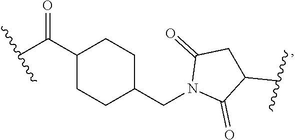 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₛ is independently a natural or non-natural amino acid. One or more AAₛ can be a natural amino acid. One or more AAₛ can be a non-natural amino acid. One or more AAₛ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

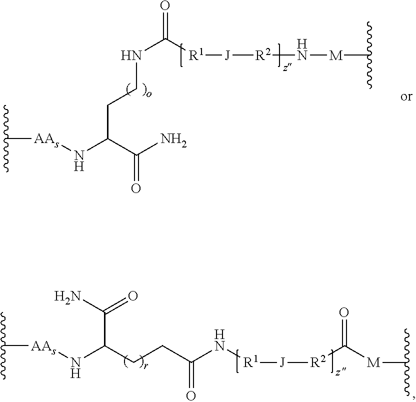

wherein M, AAₛ, each —(R¹-J-R²)z″-, o and z″ are defined herein; r can be 0 or 1.

r can be 0. r can be 1.

The linker can have the structure:

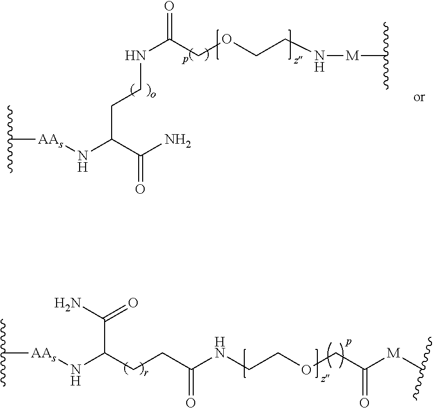

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

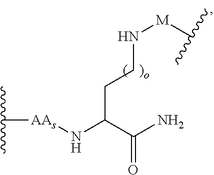

wherein:
M, AAₛ and o are as defined herein.

Other non-limiting examples of suitable linkers include:

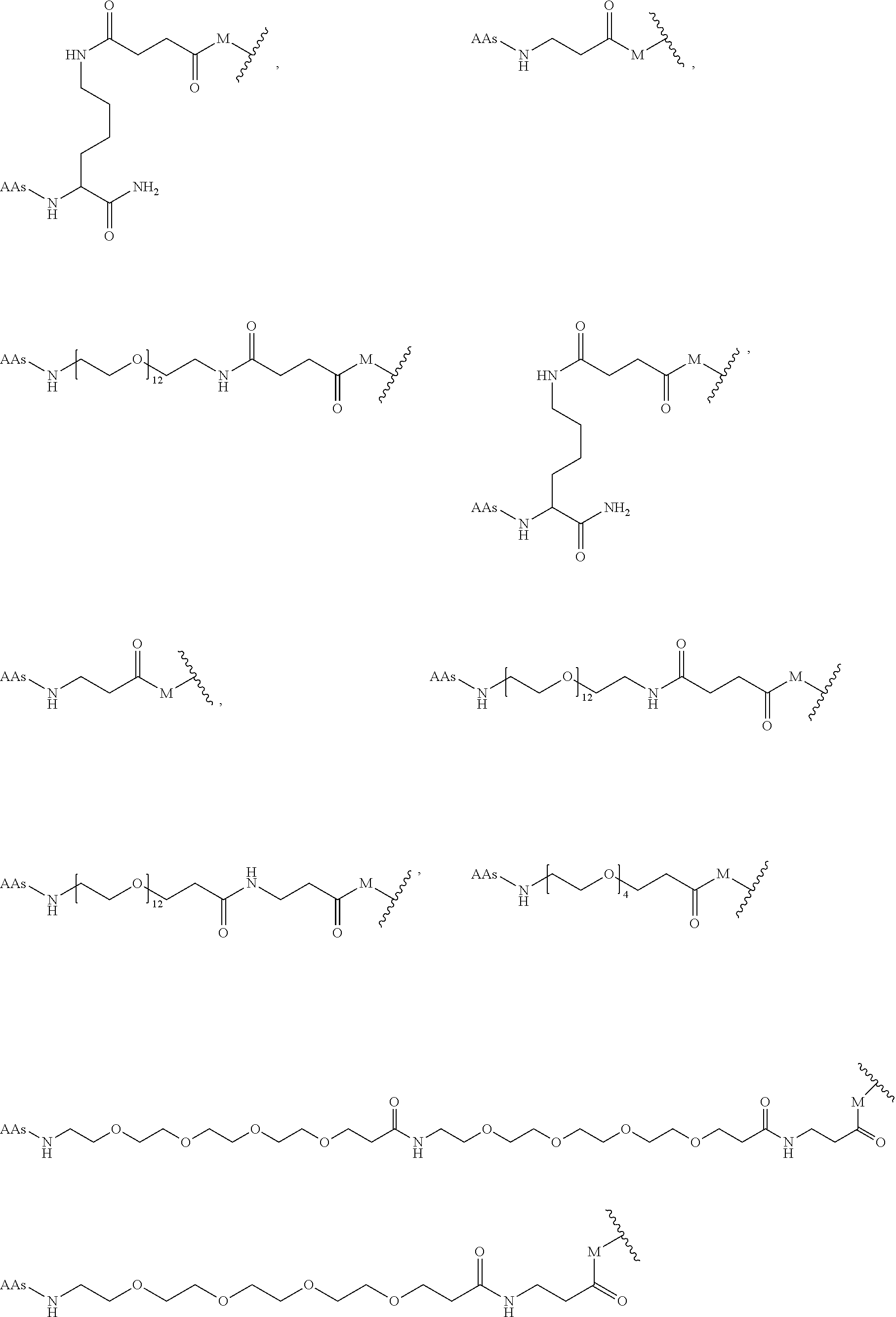

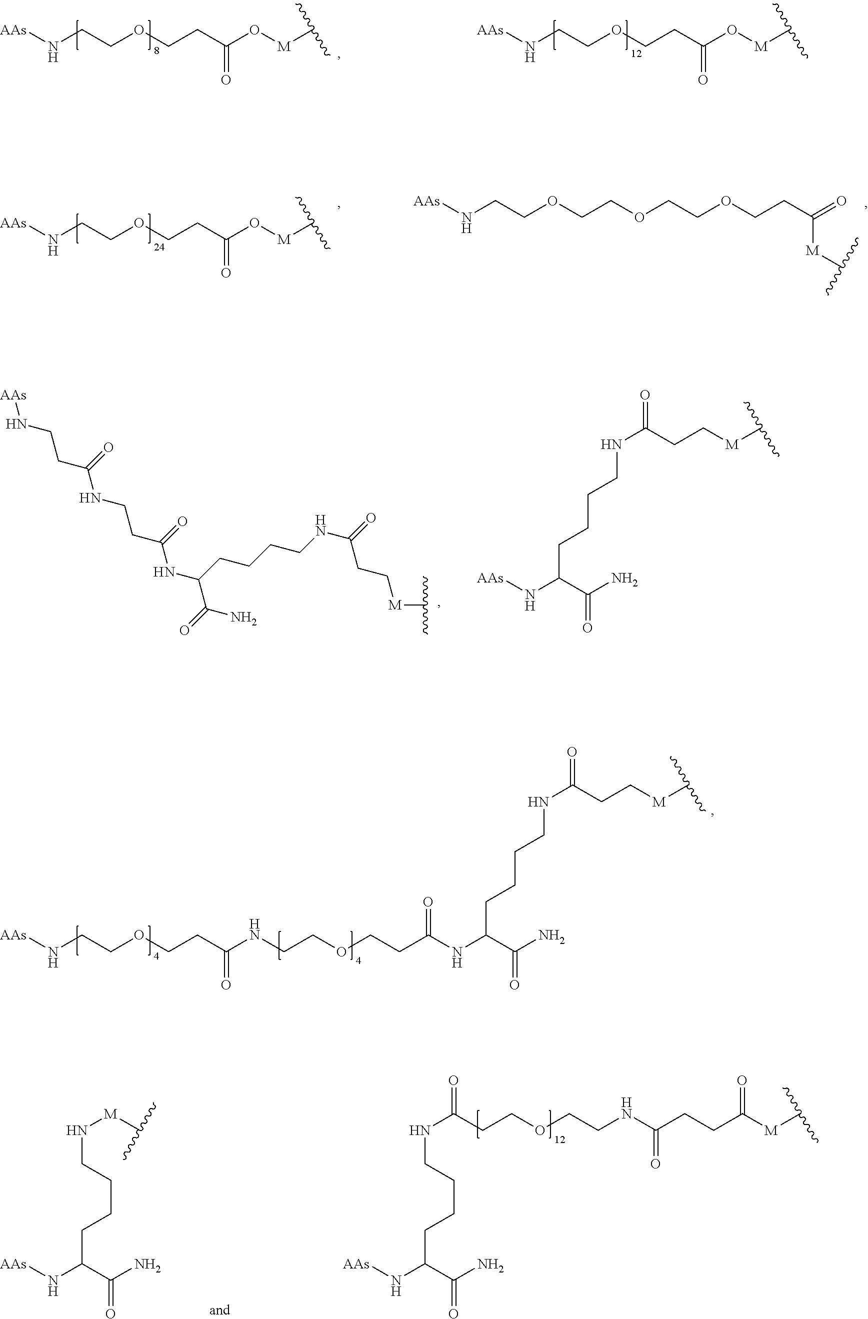

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

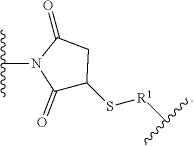

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

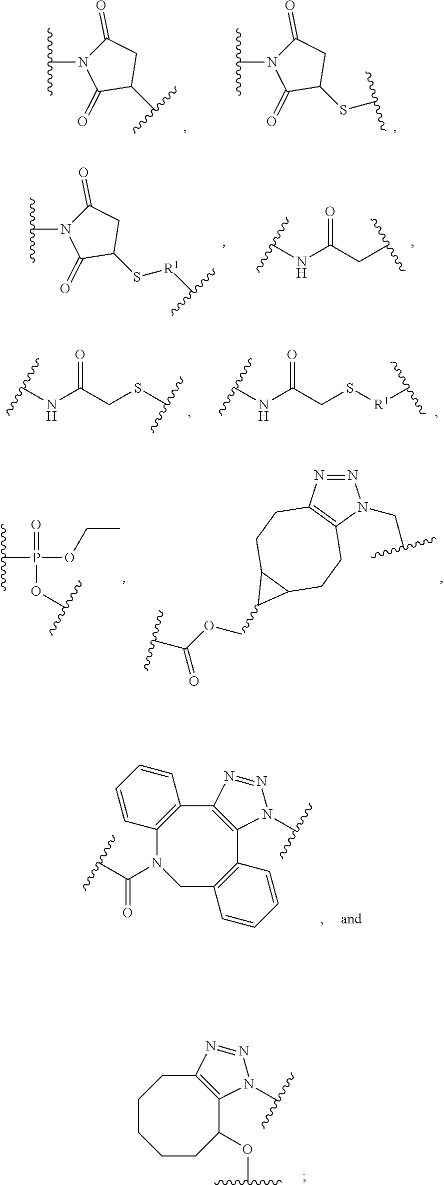

wherein: R¹ is alkylene, cycloalkyl, or

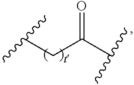

wherein t′ is 0 to 10 wherein each R is independently an alkyl, alkenyl, alkynyl, carbocyclyl, or heterocyclyl, wherein R¹ is

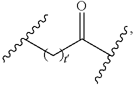

and t′ is 2.

The linker can have the structure:

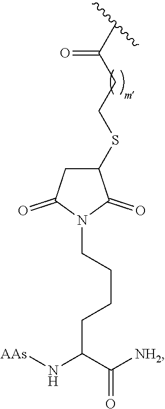

wherein AAₛ is as defined herein, and m′ is 0-10.

The linker can be of the formula:

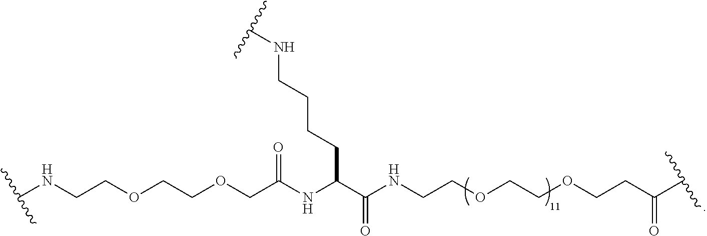

The linker can be of the formula:

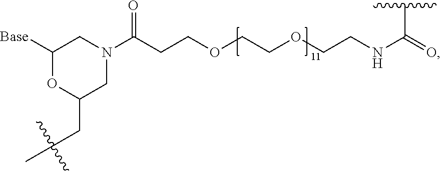

wherein “base” is corresponds to a nucleobase at the 3′ end of a cargo phosphorodiamidate morpholino oligomer.

The linker can be of the formula:

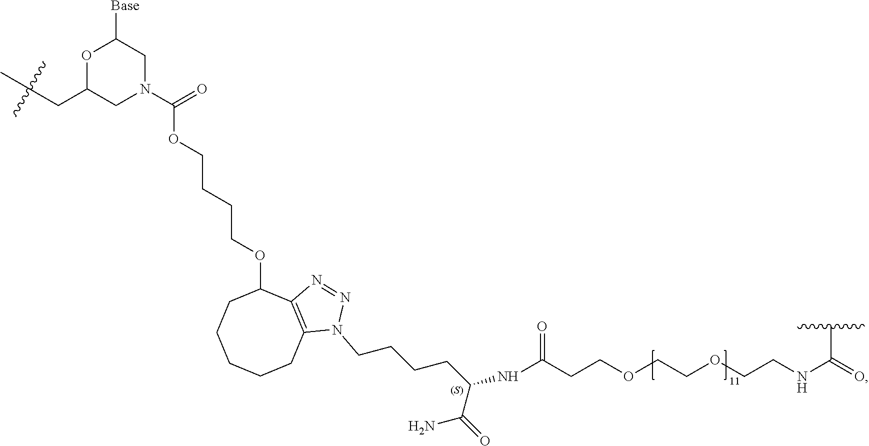

wherein “base” is corresponds to a nucleobase at the 3′ end of a cargo phosphorodiamidate morpholino oligomer.

The linker can be of the formula:

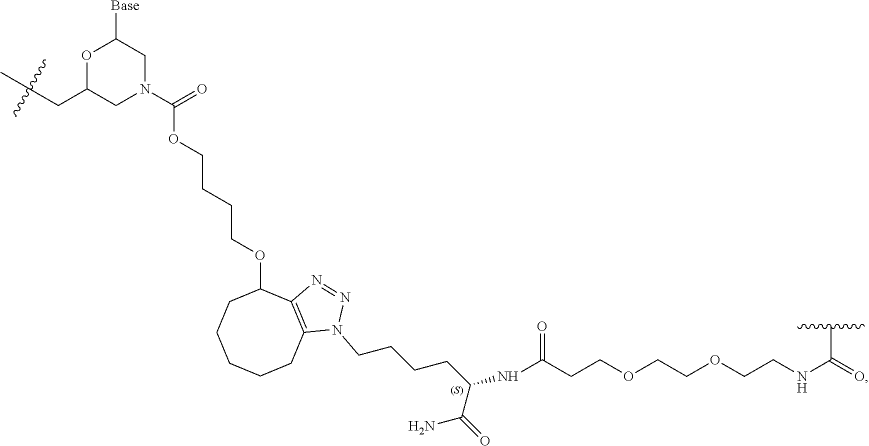

wherein “base” is corresponds to a nucleobase at the 3′ end of a cargo phosphorodiamidate morpholino oligomer.

The linker can be of the formula:

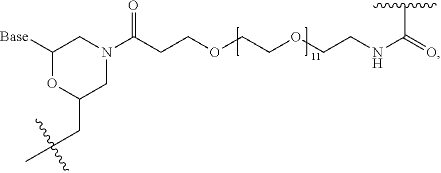

wherein “base” is corresponds to a nucleobase at the 3′ end of a cargo phosphorodiamidate morpholino oligomer.

The linker can be of the formula:

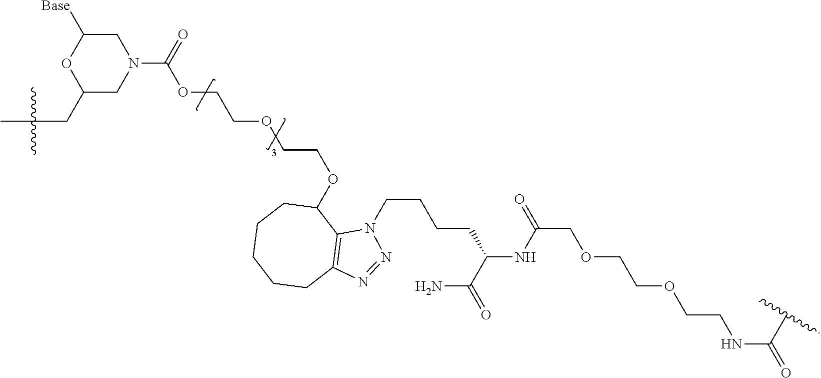

The linker can be covalently bound to a cargo at any suitable location on the cargo. The linker is covalently bound to the 3′ end of cargo or the 5′ end of an oligonucleotide cargo The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 4:

TABLE 4
cCPP

(βhFfΦGrGrQ)
(βhFfΦSrSrQ)
(βhFfΦHrHrQ)
(βhFFΦSRSRQ)
(βhFFΦGRGRQ)
(βhFFΦHRHRQ)

In embodiments, EEVs comprising a cyclic cell penetrating peptide (cCPP), linker and exocyclic peptide (EP) are provided.

In embodiments, an EEV can comprise the structure of Formula (B)

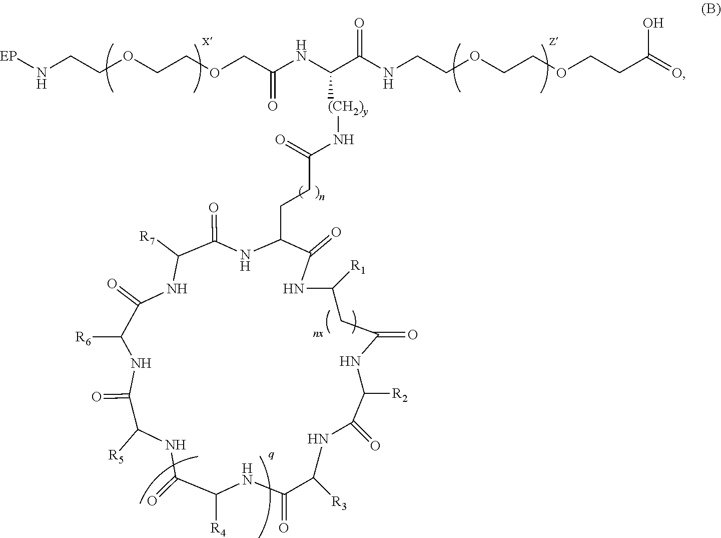

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
n is an integer from 0-2;
x′ is an integer from 1-20;
z′ is an integer from 1-23;
nₓ is 0 or 1;
y is an integer from 1-5;
q is 1, 2, 3 or 4; and
EP is an exocyclic peptide as defined herein.

In embodiments, the EEV of Formula (B) can have the structure:

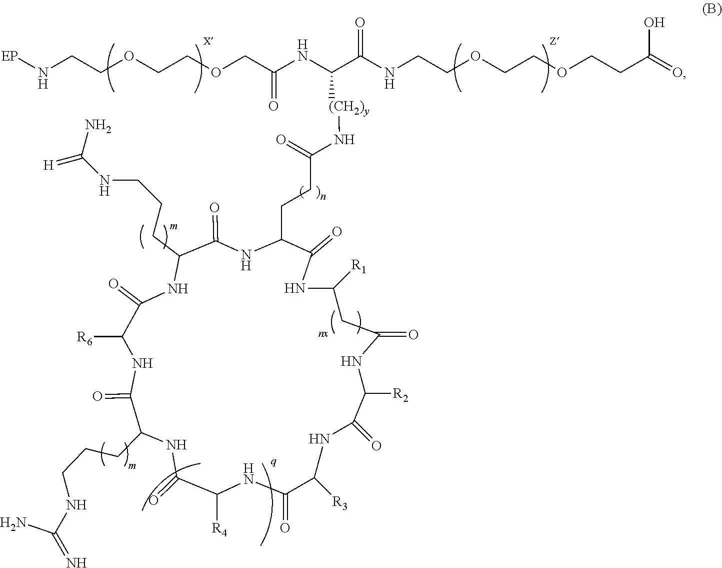

or a protonated form thereof,
wherein:
R₁, R₂, R₃, R₄, R₆, AA_{SC}, EP, n, x′, z′, y, nₓ and q are as described for Formula (B); and
each m is independently an integer from 0-3.

In embodiments, an EEV can comprise the structure of Formula (B2):

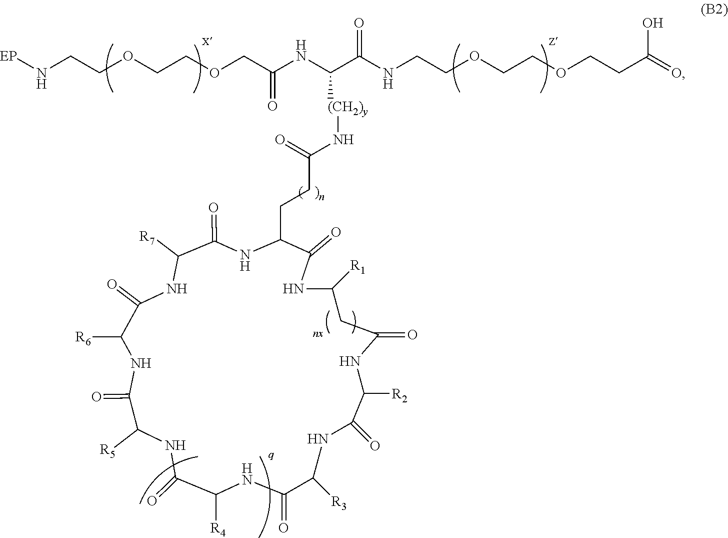

or a protonated form thereof,
wherein:
R₁, R₂, R₃, R₄, R₆, R₅, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
n is an integer from 0-2;
nₓ is 1;
x′ is an integer from 1-20;
z′ is an integer from 1-23;
y is an integer from 1-5;
q is 1, 2, 3 or 4; and
EP is an exocyclic peptide as defined herein.

In embodiments, an EEV can comprise the structure of Formula (B3):

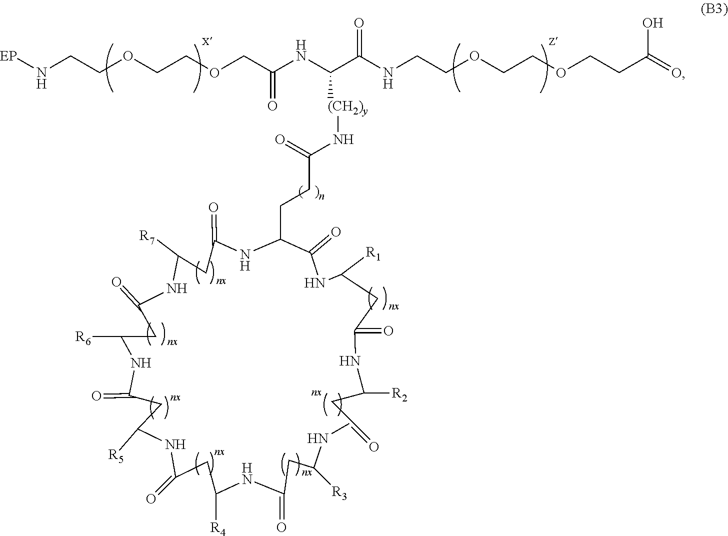

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
nₓ is 0 or 1;
only one nₓ is 1;
n is an integer from 0-2;
x′ is an integer from 1-20;
z′ is an integer from 1-23;
y is an integer from 1-5;
q is 1, 2, 3 or 4; and
EP is an exocyclic peptide as defined herein.

R₁, R₂, R₃, R₄, R₆, EP, m, q, y, x′, z′ are as described herein.

n can be 0. n can be 1. n can be 2.

nₓ can be 0. nₓ can be 1.

The EEV can comprise the structure of Formula (B-a) or (B-b):

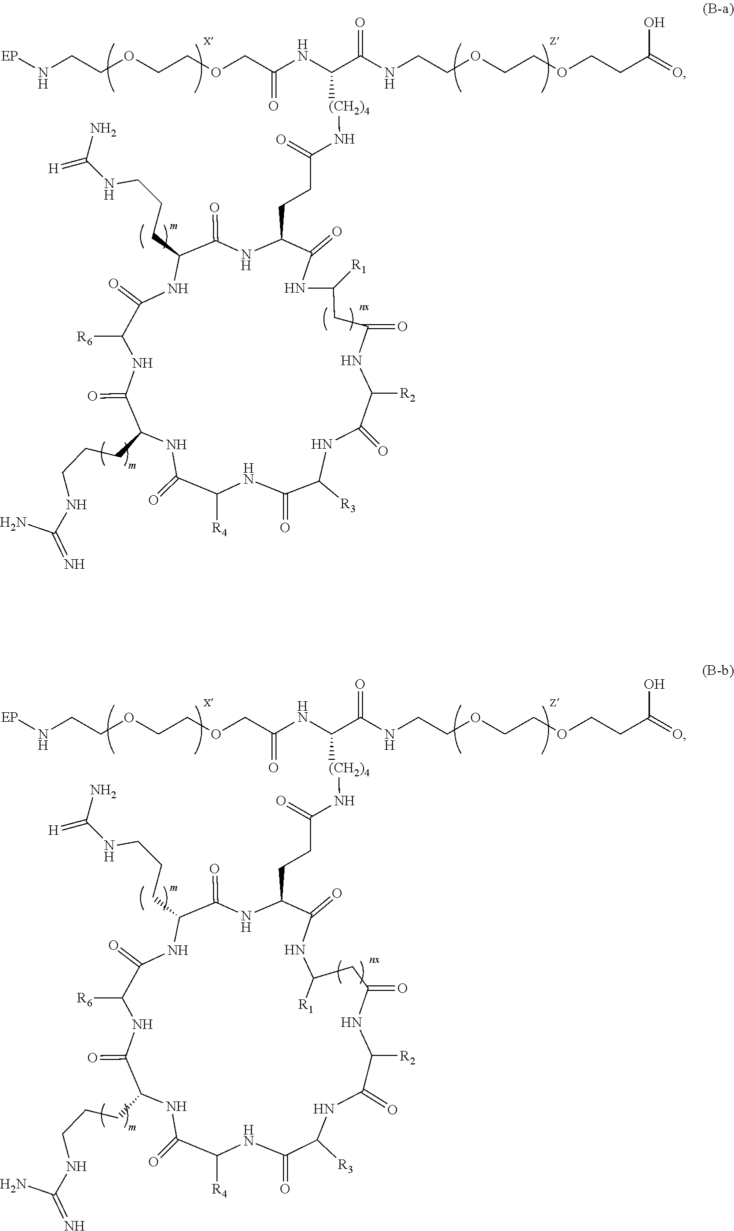

or a protonated form thereof, wherein EP, R₁, R₂, R₃, R₄, R₆, m, nₓ, x′ and z′ are as defined above in Formula (B).

The EEV can comprises the structure of Formula (B-c):

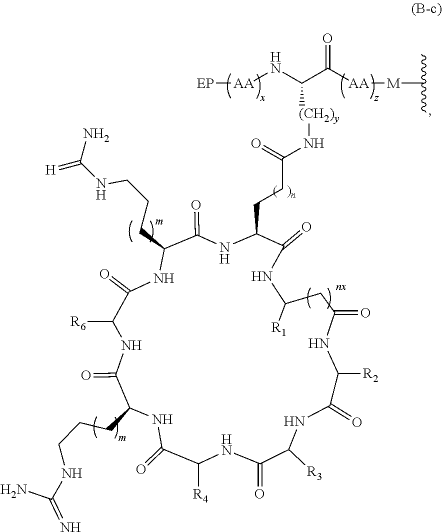

or a protonated form thereof, wherein EP, R₁, R₂, R₃, R₄, R₆, y, nₓ, and m are as defined above in Formula (B); AA is an amino acid as defined herein; M is as defined herein; n is an integer from 0-2; x is an integer from 1-10; and z is an integer from 1-10.

The EEV can have the structure of Formula:

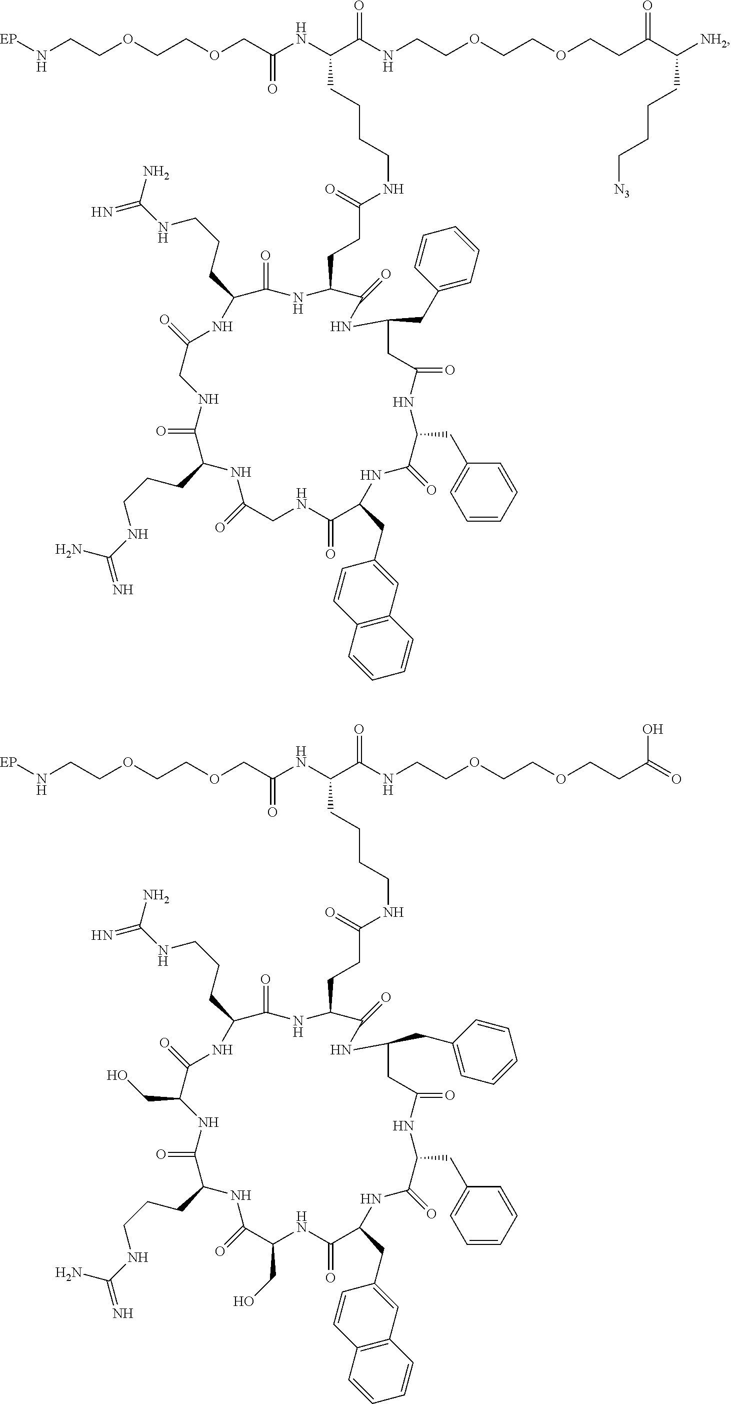

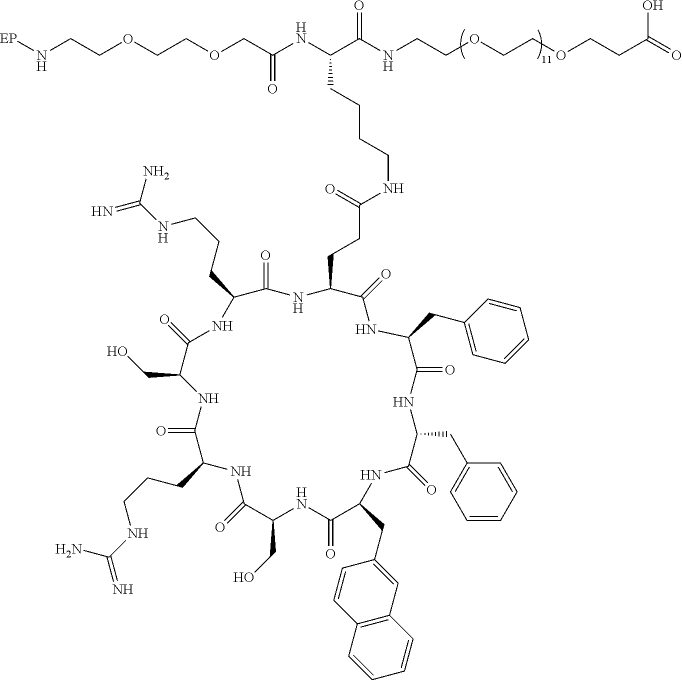

or a protonated form thereof, wherein EP is as defined above in Formula (B).

The EEV can comprise Formula (B) and can have the structure: Ac-PKKRKV-AEEA-K(cyclo[βhF-f-Φ-GrGrQ])-AEEA-K(N₃)—NH₂; Ac-PKKKRKV-AEEA-K(cyclo[Ff-Φ-SrSrQ])-AEEA-K(N₃)—NH₂, or Ac-PKKKRKV-AEEA-K(cyclo[βhF-F-Φ-SRSRQ])-PEG₁₂-OH.

The EEV can comprise two or more cCPP conjugated to the cargo. In embodiments, the EEV can be (cCCP)-linker-k(cCPP)-linker-OH. In embodiments, the compound (and EEV) may include two to more cCPPs (e.g., 2, 3, 4, 5, 6, 7, 8, 9, or 10). As such, the linker can be multivalent and link two or more cCPPs to a cargo and EP. In embodiments, the compound may include two or more linkers that allow for two or more cCPPs, one or more EPs, and one or more cargos to be linked in a single compound. For example, the compound can comprise (cCPP)-linker¹-K(cCPP)-linker²-FG where linker¹ and linker² may be distinct linkers or a single linker and FG is a functional group that is a part of a linker. The compound can comprise EP-linker¹-K(cCPP)-linker²-k(cCPP)-linker³-FG where the linkers may be distinct linkers or two or more linkers may be a part of the same linker and FG is a functional group that is a part of a linker. In embodiments, the EEV can be (cyclo[βhF-f-Φ-GrGrQ])-PEG₂-k(cyclo[βhF-f-Φ-GrGrQ])-PEG₁₂-OH. In embodiments, the EEV can be (cyclo[Ff-Φ-SrSrQ])-PEG₂-k(cyclo[Ff-Φ-SrSrQ])-PEG₁₂-OH. In embodiments, the EEV can be (cyclo[βhF-F-Φ-SRSRQ])-PEG₂-k(cyclo[βhF-F-Φ-SRSRQ])-PEG₁₂-OH.

### Cargo

The cell penetrating peptide (CPP), such as a cyclic cell penetrating peptide (e.g., cCPP), can be conjugated to a cargo. The cargo can be a therapeutic moiety. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

The cargo can comprise one or more detectable moieties, one or more therapeutic moieties, one or more targeting moieties, or any combination thereof. The cargo can be a peptide, oligonucleotide, or small molecule. The cargo can be a peptide sequence or a non-peptidyl therapeutic agent. The cargo can be an antibody or an antigen binding fragment thereof, including, but not limited to an scFv or nanobody.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to cCPP through a linker. The cargo moiety can comprise therapeutic moiety. The therapeutic moiety can comprise an oligonucleotide, a peptide or a small molecule. The oligonucleotide can comprise an antisense oligonucleotide. The cargo moiety can be conjugated to the linker at the terminal carbonyl group to provide the following structure:

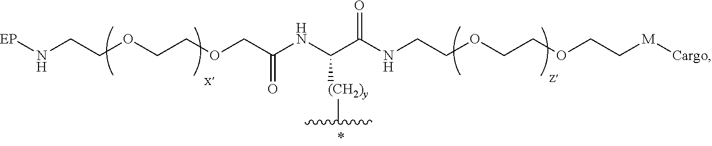

wherein:
EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

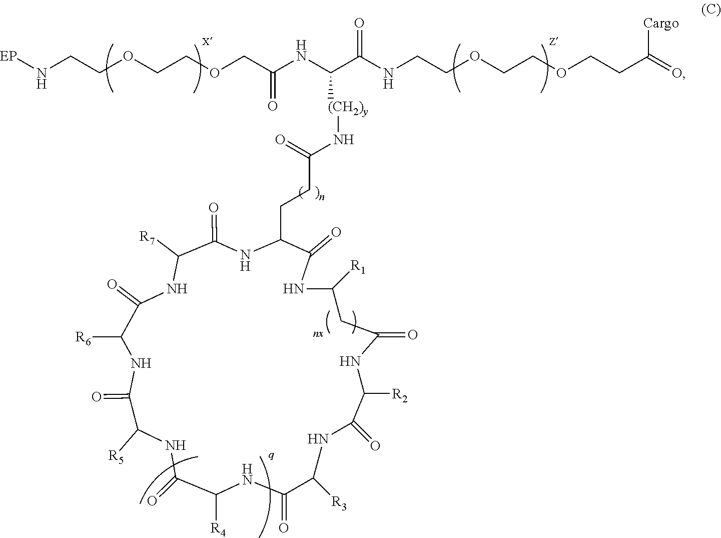

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
n is an integer from 0-2;
x′ is an integer from 1-20;
z′ is an integer from 1-23;
nₓ is 0 or 1;
y is an integer from 1-5;
q is 1, 2, 3 or 4;
EP is an exocyclic peptide as defined herein; and
Cargo is a moiety as defined herein.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C2):

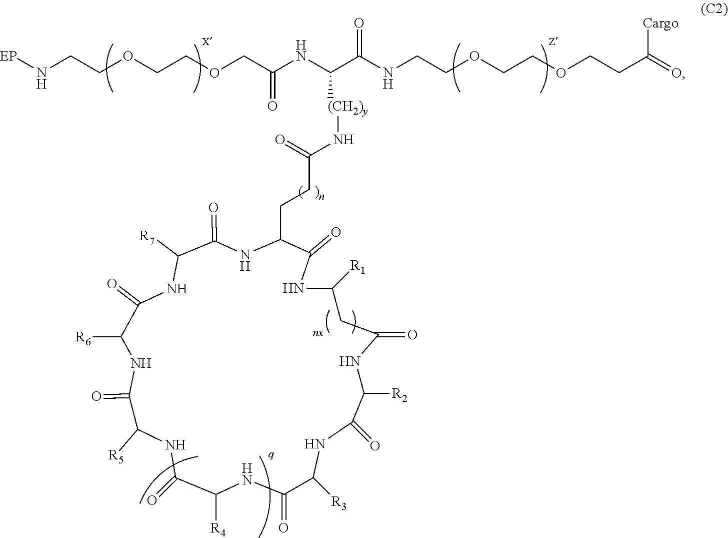

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
n is an integer from 0-2;
x′ is an integer from 1-20;
z′ is an integer from 1-23;
nₓ is 1;
y is an integer from 1-5;
q is 1, 2, 3 or 4;
EP is an exocyclic peptide as defined herein; and
Cargo is a moiety as defined herein.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C3):

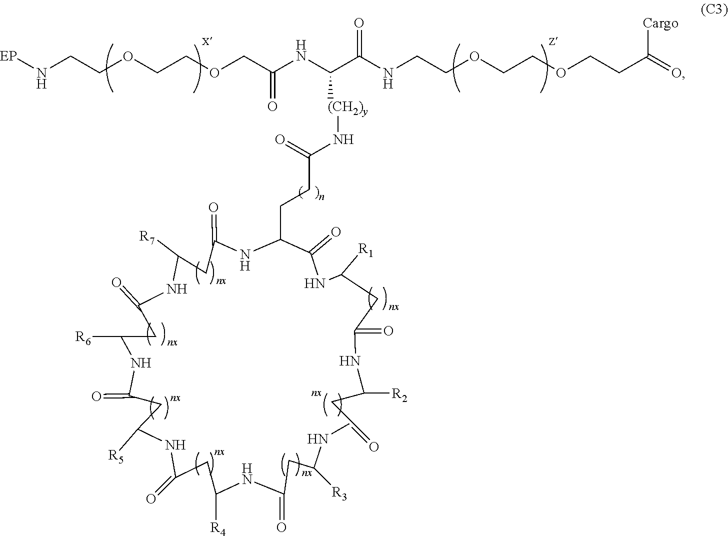

or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
nₓ is 0 or 1;
only one nₓ is 1;
n is an integer from 0-2;
x′ is an integer from 1-20;
z′ is an integer from 1-23;
y is an integer from 1-5;
q is 1, 2, 3 or 4;
EP is an exocyclic peptide as defined herein; and
Cargo is a moiety as defined herein.

The EEV-conjugate of formula (C) can be of formula:

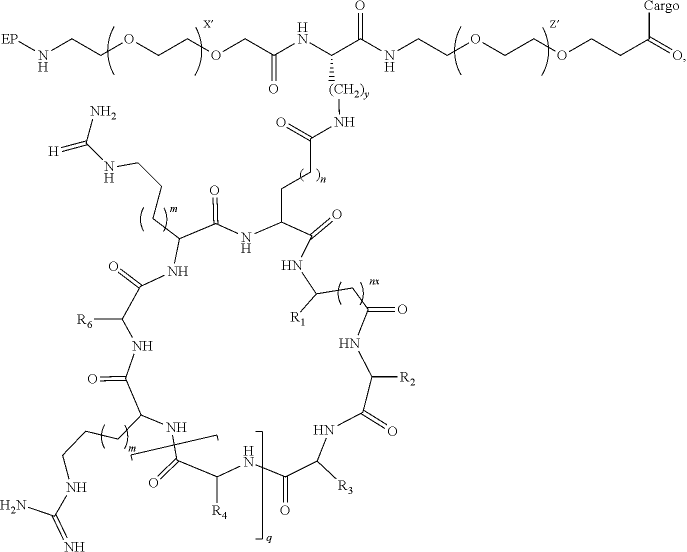

or a protonated form thereof,
wherein:
R₁, R₂, R₃ and R₇ are independently a side chain of phenylalanine, or naphthylalanine;
R₄ is H or an amino acid side chain;
EP is an exocyclic peptide as defined herein;
Cargo is a moiety as defined herein;
each m is independently an integer from 0-3;
n is an integer from 0-2;
nₓ is 0 or 1;
x′ is an integer from 1-20;
z′ is an integer from 1-23;
y is an integer from 1-5; and
q is an integer from 1-4.
R₁, R₂, R₃, R₄, R₇, EP, cargo, m, n, nₓ, x′, y, q, and z′ are as defined herein.

The EEV can be conjugated to a cargo and the EEV-conjugate can comprise the structure of Formula (C-a) or (C-b):

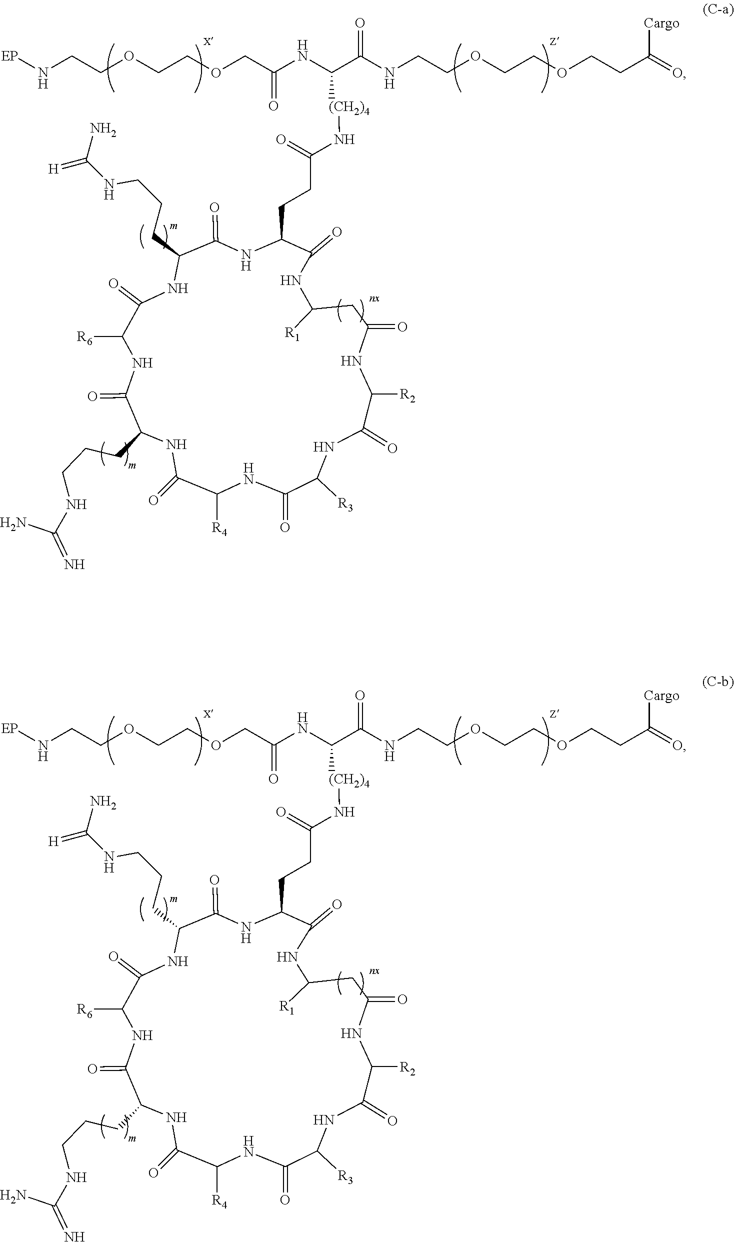

or a protonated form thereof,
wherein EP, m and z are as defined above in Formula (C) or (C2).

The EEV can be conjugated to a cargo and the EEV-conjugate can comprise the structure of Formula (C-c):

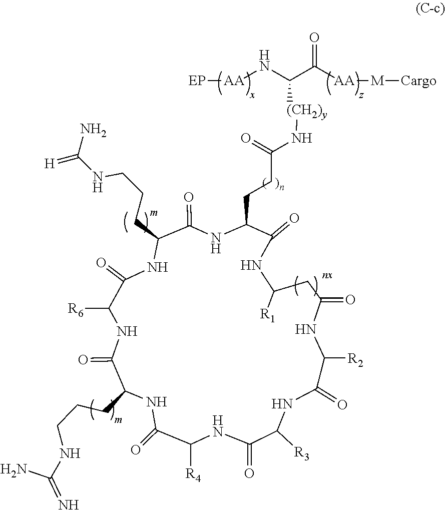

or a protonated form thereof, wherein EP, R₁, R₂, R₃, R₄, and m are as defined above in Formula (C) or (C2); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula:

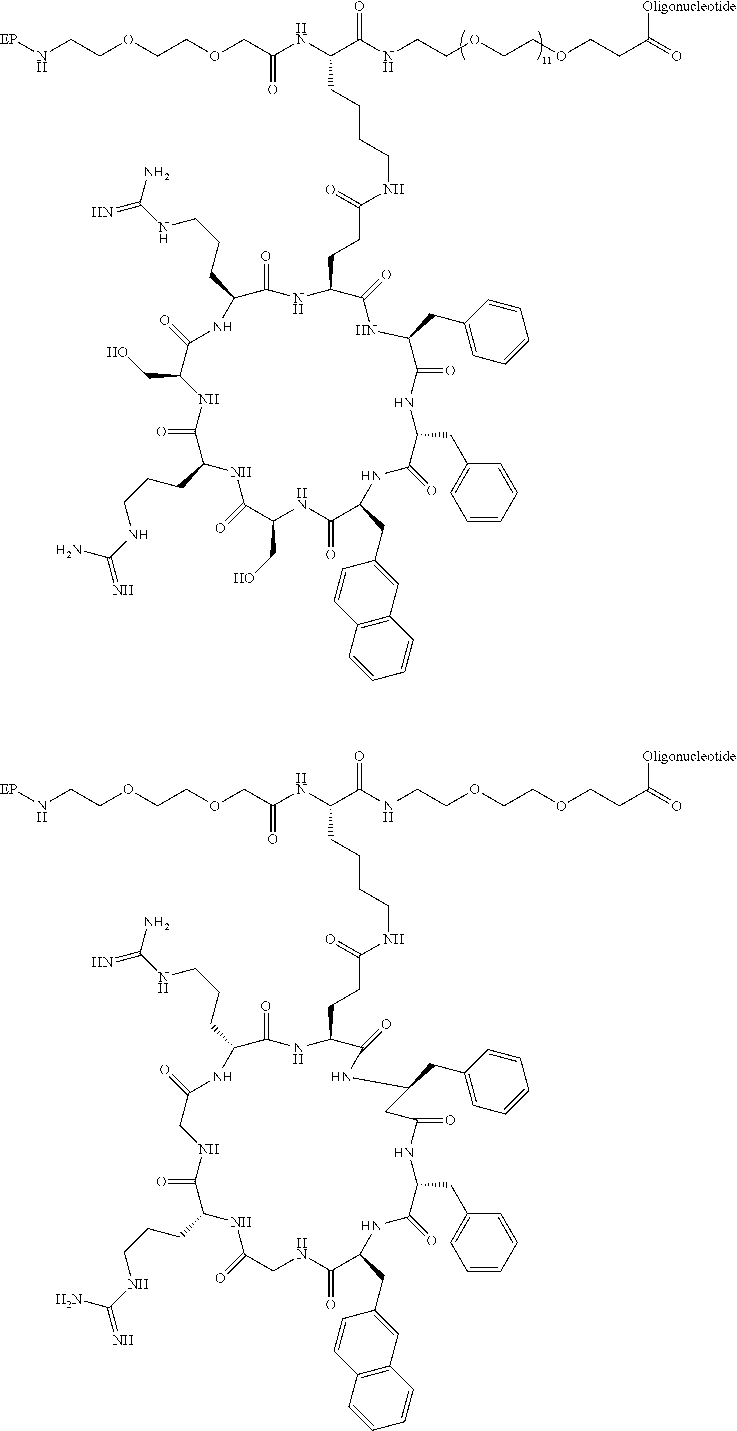

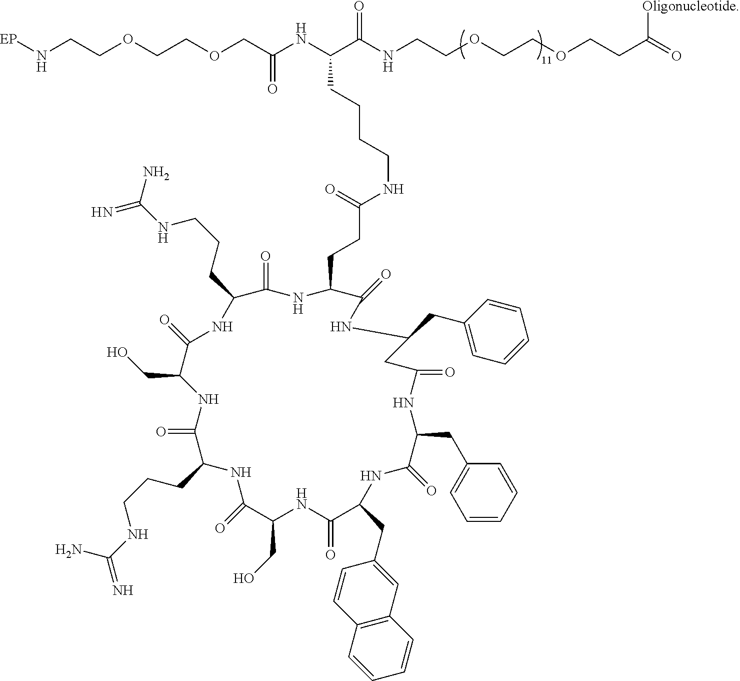

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein. The compounds of compositions can be used to treat any disease or condition that is amendable to treatment with the therapeutic moieties disclosed herein.

The compounds disclosed herein can be used for detecting or diagnosing a disease or condition in a subject. For example, a cCPP can comprise a targeting moiety and/or a detectable moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell can comprise attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. A kit can include one or more other components, adjuncts, or adjuvants as described herein. kit includes one or more anti-cancer agents, such as those agents described herein. A kit can include instructions or packaging materials that describe how to administer a compound or composition of the kit. Containers of the kit can be of any suitable material, e.g., glass, plastic, metal, etc., and of any suitable size, shape, or configuration. A compound and/or agent disclosed herein can be provided in the kit as a solid, such as a tablet, pill, or powder form. A compound and/or agent disclosed herein can be provided in the kit as a liquid or solution. A kit can comprise an ampoule or syringe containing a compound and/or agent disclosed herein in liquid or solution form.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

“AEEA”, “miniPEG”, and “PEG2” can be used interchangeably to refer to 2-[2-[2-aminoethoxy]ethoxy]acetic acid.

As used herein, the term “cyclic cell penetrating peptide” or “cCPP” refers to a peptide that facilitates the delivery of a cargo, e.g., a therapeutic moiety, into a cell.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide, peptide or small molecule) that can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV, the nucleoplasmin bipartite NLS with the sequence NLSKRPAAIKKAGQAKKKK, the c-myc nuclear localization sequence having the amino acid sequence PAAKRVKLD or RQRRNELKRSF, the sequence RMRKFKNKGKDTAELRRRRVEVSVELRKAKKDEQILKRRNV of the IBB domain from importin-alpha, the sequences VSRKRPRP and PPKKARED of the myoma T protein, the sequence PQPKKKPL of human p53, the sequence SALIKKKKKMAP of mouse c-abl IV, the sequences DRLRR and PKQKKRK of the influenza virus NS1, the sequence RKLKKKIKKL of the Hepatitis virus delta antigen and the sequence REKKKFLKRR of the mouse Mx1 protein, the sequence KRKGDEVDGVDEVAKKKSKK of the human poly(ADP-ribose) polymerase and the sequence RKCLQAGMNLEARKTKK of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic cell penetrating peptide (cCPP). The linker can comprise a natural or non-natural amino acid or polypeptide. The linker can be a synthetic compound containing two or more appropriate functional groups suitable to bind the cCPP to a cargo moiety, to thereby form the compounds disclosed herein. The linker can comprise a polyethylene glycol (PEG) moiety. The linker can comprise one or more amino acids. The cCPP may be covalently bound to a cargo via a linker.

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

The term “therapeutic polypeptide” refers to a polypeptide that has therapeutic, prophylactic or other biological activity. The therapeutic polypeptide can be produced in any suitable manner. For example, the therapeutic polypeptide may isolated or purified from a naturally occurring environment, may be chemically synthesized, may be recombinantly produced, or a combination thereof.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

As used herein, the term “contiguous” refers to two amino acids, which are connected by a covalent bond. For example, in the context of a representative cyclic cell penetrating peptide (cCPP) such as

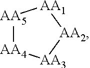

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

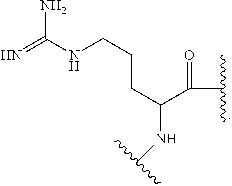 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

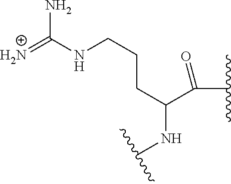 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein below.

As used herein “aromatic” refers to an unsaturated cyclic molecule having 4n+2π electrons, wherein n is any integer. The term “non-aromatic” refers to any unsaturated cyclic molecule which does not fall within the definition of aromatic. As used herein “uncharged” refers to absence of charge at physiological pH.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents. As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

What is claimed is:

1. A cell penetrating peptide of general Formula (A) (A2) or (A3): 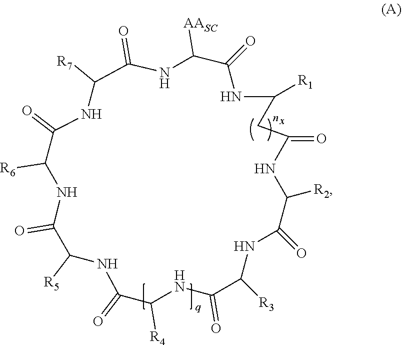
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
AA_{SC} is an amino acid side chain;
nₓ is 0 or 1; and
q is 1, 2, 3 or 4; 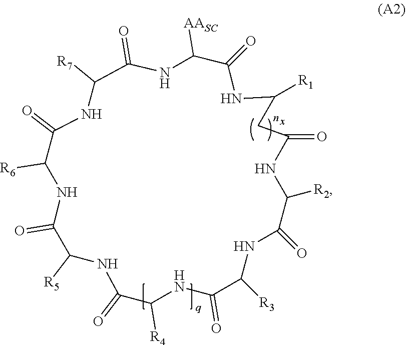
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 1; and
q is 1, 2, 3 or 4; or 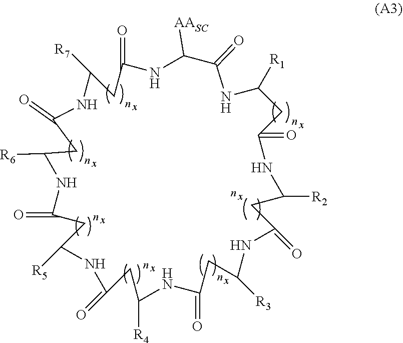
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
AA_{SC} is an amino acid side chain; and
nₓ is 0 or 1;
only one nₓ is 1; and
q is 1, 2, 3 or 4.

2. The cell penetrating peptide of claim 1, wherein in Formula (A), nₓ is 0 and q is 1.

3. The cell penetrating peptide of claim 1, wherein in Formula (A), nₓ is 1 and q is 1.

4. The cell penetrating peptide of claim 1, wherein in Formula (A2) q is 1.

5. The cell penetrating peptide of claim 1, wherein at least two of R₄, R₅, R₆, or R₇ are independently a side chain of an uncharged non-aryl amino acid selected from histidine, threonine, serine, leucine, isoleucine, valine, neopentylglycine, alanine, homoalanine, homoserine, 3-(4-thiazolyl)-alanine, 3-(4-furanyl)-alanine, and 3-(4-thienyl)-alanine.

6. The cell penetrating peptide of claim 1, wherein at least two of R₄, R₅, R₆, or R₇ are independently a side chain of serine.

7. The cell penetrating peptide of claim 1, wherein at least two of R₄, R₅, R₆, or R₇ are independently a side chain of histidine.

8. The cell penetrating peptide of claim 1, wherein cCPP of Formula (A) or (A2) can comprise the structure of Formula (I-a) or Formula (I-b): 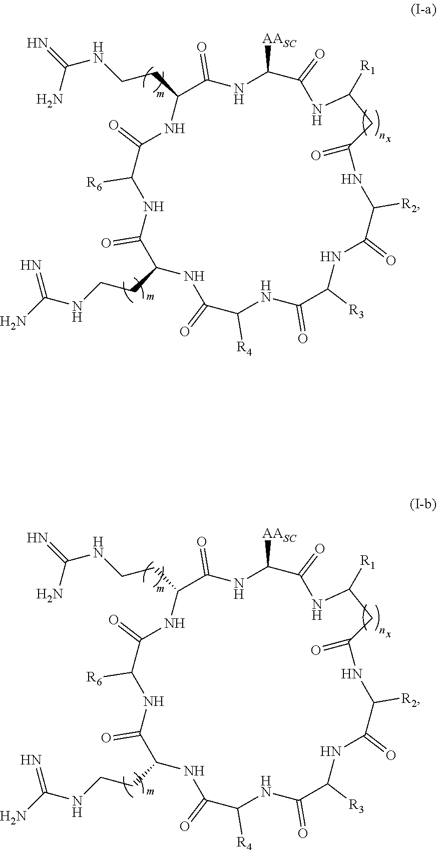
or protonated form thereof, wherein AA_{SC}, R₁, R₂, R₃, R₄, R₇, m and nₓ are as defined herein for Formula (A) or (A2).

9. The cell penetrating peptide of claim 1, wherein cCPP of Formula (A) can comprise the cCPP of Formula (A) can comprise the structure of Formula (I-4a), (I-4b), or (I-4c): 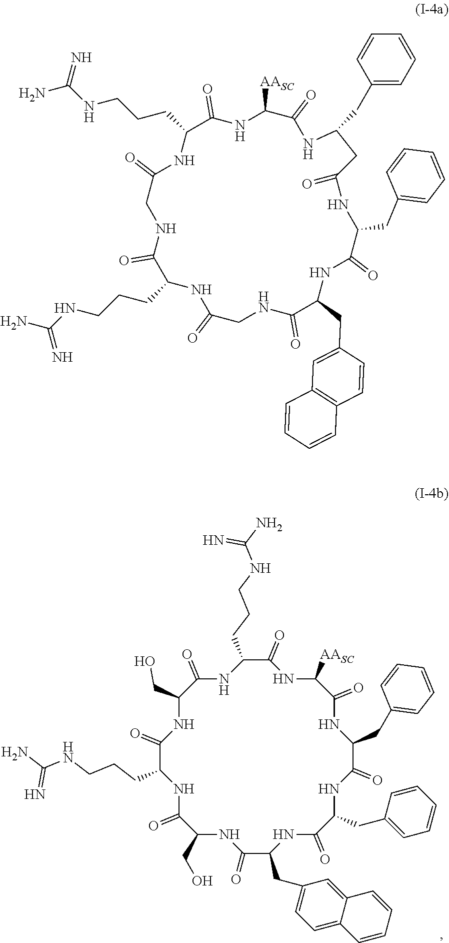 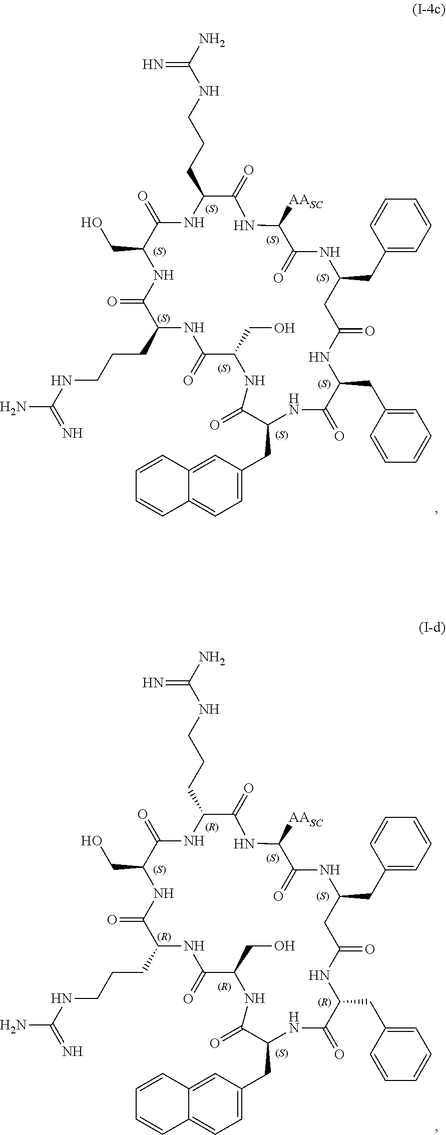
or a protonated form thereof.

10. An EEV comprising the structure of Formula (B), (B2) or (B3): 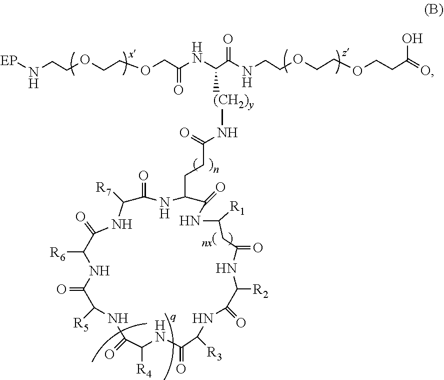
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
n is an integer from 0-2;
x′ is an integer from 1-20;
z′ is an integer from 1-23;
nₓ is 0 or 1;
y is an integer from 1-5;
q is 1, 2, 3 or 4; and
EP is an exocyclic peptide as defined herein; 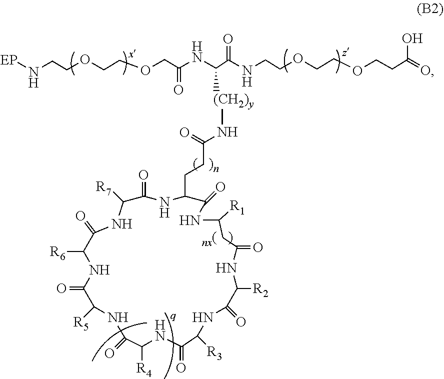
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
n is an integer from 0-2;
nₓ is 1;
x′ is an integer from 1-20;
z′ is an integer from 1-23;
y is an integer from 1-5;
q is 1, 2, 3 or 4; and
EP is an exocyclic peptide as defined herein; or 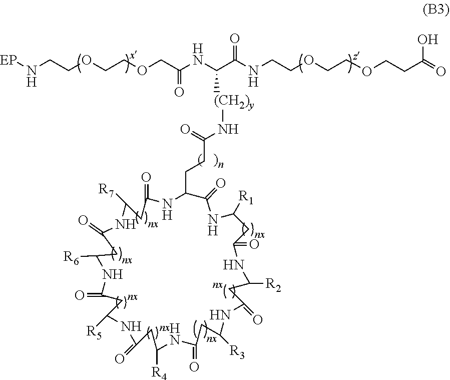
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
nₓ is 0 or 1;
only one nₓ is 1;
n is an integer from 0-2;
x′ is an integer from 1-20;
z′ is an integer from 1-23;
y is an integer from 1-5;
q is 1, 2, 3 or 4; and
EP is an exocyclic peptide as defined herein.

11. The EEV of claim 10, wherein the EEV comprises a formula selected from:

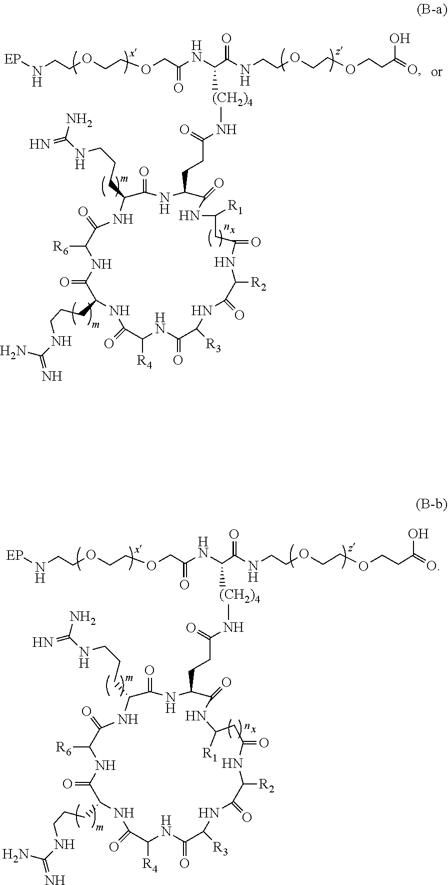

12. The EEV of claim 11, wherein nₓ is 1.

13. The EEV of claim 11 wherein x′ is 11 and z′ is 11.

14. The EEV of claim 11, wherein nₓ is 1, x′ is 11 and z′ is 11.

15. An EEV-conjugate comprising the structure of Formula (C), (C2) or (C3) 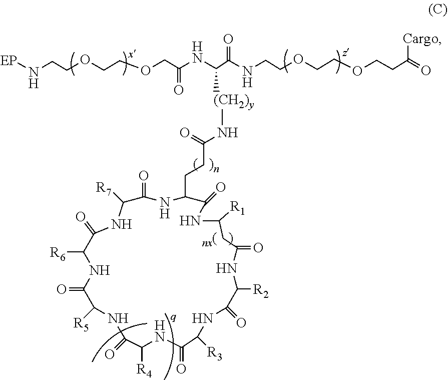
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
at least two of R₄, R₅, R₆, or R₇ are independently a side chain of serine or histidine;
n is an integer from 0-2;
x′ is an integer from 1-20;
z′ is an integer from 1-23;
nₓ is 0 or 1;
y is an integer from 1-5;
q is 1, 2, 3 or 4;
EP is an exocyclic peptide as defined herein; and
Cargo is a moiety as defined herein; 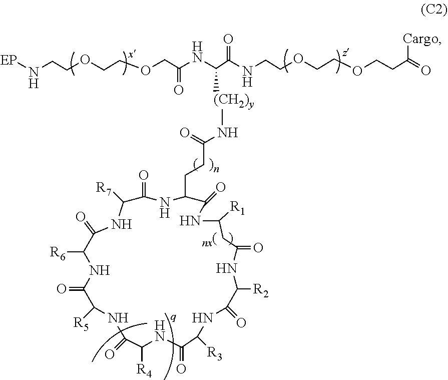
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
n is an integer from 0-2;
x′ is an integer from 1-20;
z′ is an integer from 1-23;
nₓ is 1;
y is an integer from 1-5;
q is 1, 2, 3 or 4;
EP is an exocyclic peptide as defined herein; and
Cargo is a moiety as defined herein; or 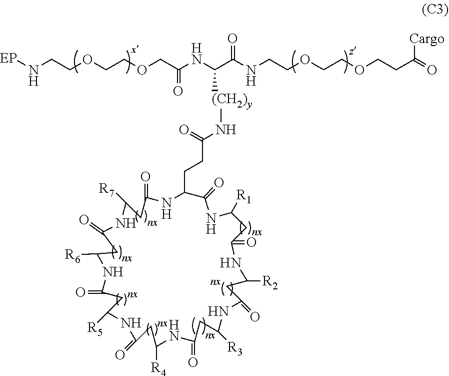
or a protonated form thereof,
wherein:
R₁, R₂, R₃, R₄, R₅, R₆, R₇ are independently H or an amino acid side chain;
at least two of R₁, R₂, and R₃ are independently a side chain of phenylalanine, or naphthylalanine;
at least two of R₄, R₅, R₆, or R₇ are independently a side chain of arginine;
nₓ is 0 or 1;
only one nₓ is 1;
n is an integer from 0-2;
x′ is an integer from 1-20;
z′ is an integer from 1-23;
y is an integer from 1-5;
q is 1, 2, 3 or 4;
EP is an exocyclic peptide as defined herein; and
Cargo is a moiety as defined herein.

16. The EEV-conjugate of claim 15, wherein the EEV-conjugate is of formula: 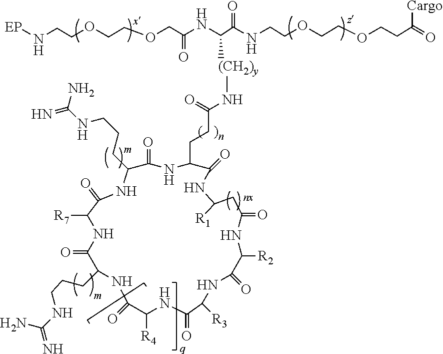
or a protonated form thereof,
wherein:
R₁, R₂, R₃ and R₇ are independently a side chain of phenylalanine, or naphthylalanine;
R₄ is H or an amino acid side chain;
EP is an exocyclic peptide as defined herein;
Cargo is a moiety as defined herein;
each m is independently an integer from 0-3;
n is an integer from 0-2;
nₓ is 0 or 1;
x′ is an integer from 1-20;
z′ is an integer from 1-23;
y is an integer from 1-5; and
q is an integer from 1-4.

17. The EEV-conjugate of claim 15, wherein the EEV-conjugate is of formula: 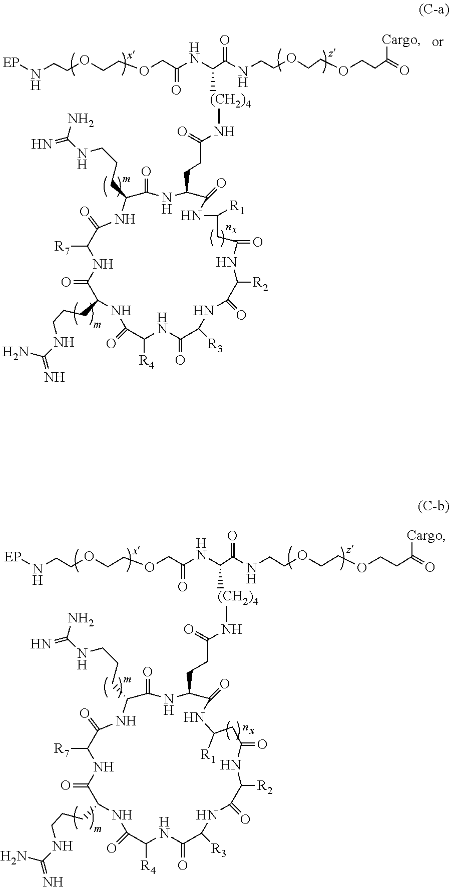
or a protonated form thereof

18. The EEV-conjugate of claim 17, wherein nₓ is 1.

19. The EEV-conjugate of claim 17, wherein x′ is 11 and z′ is 11.

20. The EEV-conjugate of claim 17, wherein nₓ is 1, x′ is 11 and z′ is 11.
